(19) United States
(12) Patent Application Publication
(10) Pub. No.: US 2015/0073232 A1
(43) Pub. Date: Mar. 12, 2015

(54) DEVICES AND METHODS FOR AIRFLOW DIAGNOSIS AND RESTORATION

(71) Applicant: Intelligent Widgets, Inc., Dublin, CA (US)

(72) Inventors: Waseem Ahmad, Union City, CA (US); Waseem A. Shaikh, Dublin, CA (US); Aamir A. Faruqui, Danville, CA (US); Ali Israr, Monroeville, PA (US); Abdul Rahim Khatri, San Ramon, CA (US); Sohaib Shaikh, New Hartford, NY (US)

(73) Assignee: Intelligent Widgets, Inc., Dublin, CA (US)

(21) Appl. No.: 14/245,414

(22) Filed: Apr. 4, 2014

Related U.S. Application Data
(60) Provisional application No. 61/827,745, filed on May 27, 2013.
(60) Provisional application No. 61/827,744, filed on May 27, 2013.
(60) Provisional application No. 61/809,060, filed on Apr. 5, 2013.
(60) Provisional application No. 61/808,958, filed on Apr. 5, 2013.
(60) Provisional application No. 61/808,990, filed on Apr. 5, 2013.
(60) Provisional application No. 61/808,937, filed on Apr. 5, 2013.
(60) Provisional application No. 61/808,952, filed on Apr. 5, 2013.

(51) Int. Cl. A61F 5/56; A61B 5/0402; A61N 1/36; A61B 5/00; A61B 5/11; A61B 5/1455; A61B 7/00; A61B 5/085

(52) U.S. Cl. CPC A61F 5/56; A61B 7/003; A61B 5/0402; A61B 5/085; A61B 5/4812; A61B 5/11; A61B 5/14551; A61B 5/6822; A61B 5/4818; A61N 1/3601
USPC 600/301; 128/848; 607/42

(57) ABSTRACT

Devices for monitoring patient breaching comprise a collar having a microprocessor and memory which is connectable to a plurality of sensors. Therapeutic devices comprise similar diagnostic capabilities and further provide energy delivery elements for stimulating a patient's upper respiratory muscles in order to terminate and an apneic or snoring event.

### CROSS-REFERENCE TO RELATED APPLICATIONS

This application claims the benefit of the following provisional patent applications, the full disclosures of which are incorporated herein by reference: 61/827,745, filed May 27, 2013; 61/827,744, filed May 27, 2013; 61/809,060, filed Apr. 5, 2013; 61/808,958, filed Apr. 5, 2013; 61/808,990, filed Apr. 5, 2013; 61/808,952, filed Apr. 5, 2013; and 61/808,937, filed Apr. 5, 2013.

### BACKGROUND OF THE INVENTION

1. Field of the Invention

The present invention relates generally to medical devices and methods. More particularly, the present invention describes an externally positioned device for monitoring, diagnosing and optionally treating snoring and sleep apnea.

Snoring is very common among mammals including humans. Snoring is a noise produced while breathing during sleep due to the vibration of the soft palate and uvula. Not all snoring threatens health, but even mild snoring can bother a bed partner or others near the person who is snoring. If the snoring gets worst overtime and goes untreated, it could lead to apnea which is a much more serious problem.

Those with apnea stop breathing in their sleep, often hundreds of times during the night. Usually apnea, referred to as obstructive sleep apnea (OSA), occurs when the throat muscles and tongue relax during sleep and partially block the opening of the airway. When the muscles of the soft palate at the base of the tongue and the uvula relax and sag, the airway becomes blocked, making breathing labored and noisy and even stopping it altogether. Sleep apnea also can occur in obese people when an excess amount of tissue in the airway causes it to be narrowed. In a given night, the number of involuntary breathing pauses or “apneic events” may be as high as 20 to 60 or more per hour. These breathing pauses are almost always accompanied by snoring between apnea episodes. Sleep apnea can also be characterized by choking sensations.

Sleep apnea is diagnosed and treated by primary care physicians, pulmonologists, neurologists, or other physicians with specialty training in sleep disorders. Diagnosis of sleep apnea is not simple because there can be many different reasons for disturbed sleep. Patients are usually evaluated based on medical history, physical examination, and testing such as polysomnography. Testing must often be performed in a “sleep laboratory,” requiring the patient to spend a night in a medical facility often wired to a variety of different diagnostic machines.

Once the condition has been diagnosed, a variety of therapies are available for treating snoring and/or sleep apnea. Currently available therapies include nasal continuous positive airway pressure (CPAP), which is the most common treatment for sleep apnea. In this procedure, the patient wears a mask over the nose during sleep, and pressure from an air blower forces air through the nasal passages. While often very effective, the need to wear a mask all night is unacceptable to many and at least discomforting to most. Dental appliances that advance the mandible (lower jaw) and the tongue are less obtrusive that CPAP masks and are helpful to a limited percentage of patients with mild to moderate sleep apnea or who snore but do not have apnea. In serious cases of apnea, surgery may be required. Uvulopalatopharyngoplasty (UPPP) is a conventional surgical procedure used to remove excess tissue at the back of the throat (tonsils, uvula, and part of the soft palate). Laser-assisted uvulopalatoplasty (LAUP) is a “minimally invasive” surgical procedure used to shrink tissue and to eliminate snoring but has not been shown to be effective in treating sleep apnea. Such surgical procedures, and others such as tracheostomy and somnoplasty, have varying levels of success and all have the risks associated with surgical interventions.

U.S. Pat. No. 5,123,425, teaches a particular device and method which addresses certain of the shortcomings noted above. The '425 patent describes a “sleep apnea collar” which is worn around the patient's neck and carries one or more sensors for monitoring breathing. When an apenic event is detected, electrodes on the collar deliver current to the genioglossus or other muscles to cause the muscle to contract to clear the upper airway and relieve the apnea. While promising in theory, variations in patient anatomy make such “transcutaneous” muscle stimulation difficult to control. In particular, to assure that the current is able to stimulate the muscles, the current level must be set so high that it exceeds the “arousal threshold” which will wake the patient. Even if the current is adjusted by “trial-and-error,” a level that is effective at one time will often be ineffective at other times, for example as a result of changes in patient position, tissue moisture (and hence tissue resistivity), and the like.

For these reasons, it would be desirable to provide improved devices and methods for both diagnosing and treating snoring and sleep apnea. The methods and devices should be non-surgical requiring no invasive or minimally invasive interventions and should avoid the need for the patient to wear a device in or over the mouth. The diagnostic methods and devices should be able to detect and collect a wide variety of patient and environmental conditions which can be correlated with snoring and apnea. The therapeutic methods and devices should be self-adjusting so that a treatment level can be periodically or continuously adjusted to assure effectiveness in snoring/apnea cessation while remaining below the arousal threshold for the patient. At least some of these objectives will met by the inventions described below.

2. Description of the Background Art

U.S. Pat. No. 5,123,425, has been described above. Other patents and publications of interest include: U.S. Pat. Nos. 8,626,281; 8,359,108; 8,359,097; 8,348,941; 8,326,429; 8,326,428; 8,276,585; 8,272,385; 8,249,723; 8,244,359; 8,220,467; 8,160,712; 7,720,541; 7,155,278; 6,290,654; and 5,265,624; and U.S. Pat. Publ. Nos. 2012/0071741; 2008/0243017; 2008/0243014; and 2006/0155205, the full disclosures of which are incorporated herein by reference.

### SUMMARY OF THE INVENTION

The present invention provides an airflow diagnostic and/or restoration device to diagnose and/or treat obstructive sleep apnea (OSA). A diagnostic product will be particularly useful for home sleep testing but will also find use in clinical and other settings. A therapeutic product will have both OSA detection and treatment capabilities. Both devices are reusable products, typically including some disposable components. In exemplary embodiments, the device is worn around the neck of the patient. In most cases the devices can be placed and removed by patient without requiring any assistance. A particularly useful design is a collar in the form of a C-clamp that can be placed on and removed from the patient's neck using a single hand. When worn, the device is comfortable to maximize patient compliance. The device is completely non-invasive and can be used with minimal preparation.

The therapeutic device differs from the diagnostic device primarily in the inclusion of a low intensity transcutaneous electrical muscle stimulation (EMS) capability, typically using two or more stimulation electrodes or pads incorporated into the collar or other component worn by the patient, typicall around or near the neck. Upon detecting an airflow disruption, the device generates a calibrated EMS stimulation and delivers it to the patient. The EMS stimulation typically comprises a stimulatory electrical pulse delivered to muscles of the upper airway, typically muscles in the upper airway or throat such as the genioglossus muscle, where such stimulation opens the patient's airway a small amount, typically a few millimeters, to restore airflow and reduce or eliminate snoring and apnea. Advantageously, by continuing to monitor the symptom(s) in real time, stimulation can be stopped upon detecting resumption of normal breathing. Further advantageously, stimulation intensity can be continuously adjusted in real time to restore normal breathing without exceeding an “arousal threshold” which would wake or otherwise disturb the patient. In a typical protocol, stimulation intensity can be initiated at a level well below the expected arousal threshold and, if necessary, increased in small steps until normal breathing is restored.

Real time monitoring and data collection provide a number of advantages. By collecting patient symptoms and ambient conditions in real time and over extended periods, data can be correlated with the onset of snoring and apneic events, allowing early and predictive stimulation, i.e. respiratory muscle stimulation can in some cases be commenced even before snoring or an apneic event begin. Additionally, the level and type of therapy which are effective for a particular patient can be determined by observing the correlations over time, allowing the system to begin an intervention with an amount of stimulation predicted to be sufficient to open the patient's airway and restore breathing with exceeding that patient's arousal threshold.

In a first aspect of the present invention, a device for collecting sleep data from a patient comprises a component wearable by the patient. Exemplary wearable components include collars, bands, straps, hats, vests, visors, necklaces and other platforms, housings, frames, or like, which may be worn by the patient on or near the neck in order to establish proximity to both the oral and nasal cavities in order to detect symptoms of snoring and sleep apnea. The wearable component will carry a microprocessor, memory, and a power source, and the device will further comprise a plurality of at least two sensors connectable to the microprocessor. The sensors are typically selected from the group consisting of (a) a microphone for detecting tracheal sounds, (b) a microphone for detecting snoring sounds, (c) a microphone for detecting ambient sounds, (d) a pulse oximeter, (e) a body position sensor, (f) a body motion sensor, (g) a breathing effort sensor, (h) ECG electrodes, (i) sleep stage sensors, (j) a muscle tone sensor, and the like. The microprocessor is configured to analyze and/or store in the memory at least a portion of the data collected by the sensors and delivered to the microprocessors. In exemplary embodiments, the device will comprise at least three sensors, usually comprising at least four sensors, still more usually at least five sensors, and often comprising at least six sensors, at least seven sensors, and frequently comprising all eight of listed sensors. Other sensors may also be included.

In particular embodiments, at least some of the sensors will be disposed on the wearable component, and in other embodiments at least some of the sensors will be located remotely and be connected to the component by connector element(s), including both wired and wireless connector elements. Usually, the devices will have sensors which are both mounted on the component and located remotely from the component. Also preferably, the sleep data collection devices as set forth above, will typically be used in combination with a remote storage and/or analytical device (referred to as a remote storage device) which can receive at least some of the data collected by the collection device. Remote storage and/or analytical devices may take a variety of forms, conveniently being a smart phone, personal digital assistant, or other personal communication device which can be carried by the patient and which can be connected to the collection device either via wires or wirelessly. Still further optionally, the remote storage device may further communicate with a central storage/analytical location or other system elements which are capable of receiving and storing and/or analyzing the data transmitted by the remote storage device. In such cases, the central storage/analytical location will be capable of transmitting information back to the remote storage and/or analytical device and optionally to the collection device, either directly or through the remote storage and/or analytical device.

In a second aspect of the present invention, a method for collecting sleep data from a patient comprises placing a component on the patient where the component carries a microprocessor, memory and a power source. The method may utilize any of the component configurations described above.

The method further may further comprise collecting data relating to at least two symptoms of the patient, where the symptoms may include any two or more of (a) tracheal sounds, (b) snoring sounds, (c) ambient sounds, (d) blood oxygen saturation, (e) body position, (f) breathing effort, (g) ECG, (h) sleep stage, (i) muscle tone, and the like. Usually, the methods will collect at least three of these symptoms, more usually at least four of these symptoms, still more usually at least five of the symptoms, and often at least six, seven, or all eight of the symptoms from the list.

As with the devices described above, the methods will place the component around or near the neck, and the data will be collected with sensors which are connected to deliver data to the microprocessor. Typically, at least some of the sensors will be located on the component, while often at least some of the sensors will be disposed remotely from the component. The methods may further provide for transmitting the collected data to a remote storage and/or analytical device, such as a smart phone or other personal device carried by the patient. The smart phone or other local device may further transmit the information to a central storage and/or analytical location, and information may also be transmitted back from the central storage and/or analytical location to either or both of the local storage device and the data collection device in order to better control operation of the system.

In a third aspect of the present invention, a device for restoring airflow in a sleeping patient comprises a component wearable by the patient. The wearable component may take any of the forms described above in connection with the sleep data collection device, and will similarly have a microprocessor, memory, circuitry, and a power source located thereon.

The device for restoring airflow will usually not be intended for principally diagnosing apnea or other breathing and sleeping disorders, and will usually not require as many sensors as will be useful on the diagnostic device. Thus, the therapeutic device can comprise only a single sensor, but will often comprise two, three, four, or more of the sensors described above with reference to the diagnostic device. In particular, the therapeutic device will include sensors capable of detecting the onset of an apneic or snoring event, such as microphones for detecting tracheal and snoring sounds as well as pulse oximeters, body position sensors, and the like, which are useful in performing predictive analysis of the patient's sleep condition in order to determine when an apneic or snoring event may occur.

Also, unlike the diagnostic devices, the therapeutic devices will include an output element in order to deliver a treatment to the patient in order to terminate or alleviate the snoring and/or apneic event. Typically, the output elements comprise of one or more electrical delivery elements, such as gel or other electrode pads, which can be attached to the patient's skin in order to transcutaneously deliver current to target muscles of the upper airway, particularly the genioglossus or other upper airway muscle which can be contracted to alleviate snoring and apneic events.

The microprocessor of the therapeutic device is configured to control at least one property of an electrical output, where the property may be selected from the group consisting of current, voltage, frequency, pulse repetition pattern, pulse width, duty cycle, waveform, and the like. The microprocessor and the memory are further configured to monitor and store data representing correlations between the delivered outputs and the restoration of normal airflow. Typically, the collected data may be transmitted to a remote storage and/or analytical device (referred to as a remote storage device) and optionally the remote storage device may be configured to deliver the data to a central storage and/or analysis location, either or both of which can store and further analyze the data in order to correlate the data to provide a baseline for that patient. The baseline provides a relationship between (1) patient and ambient conditions and (2) the likelihood of onset of an apneic or snoring event. Using the baseline, treatment can be initiated at a level predicted to prevent or terminate an apneic or snoring event based on the patient and/or ambient conditions. The level or type of stimulation delivered to the patient to treat the snoring or apneic event can also be “titrated” by initiating therapy at a relatively low level and, in the absence of a positive response, increasing that level until storing or the apneic event are alleviated or terminated. In this way, treatment of these events can be achieved with a reduced likelihood that the treatment will exceed an arousal threshold for the patient.

In a forth aspect of the present invention, a method for restoring airflow in a sleeping patient comprises monitoring at least one symptom of airflow disruption while the patient is sleeping. An initial stimulating energy may be applied to a muscle of the patient's upper airway, such as the genioglossus muscle, when a symptom of airflow disruption is detected. The symptoms(s) continue to be monitored, and it is determined whether the symptom of airflow disruption has been alleviated in response to the initial energy stimulation. If not, additional or alternative forms of stimulating energy can be delivered to the muscle, where the additional/alternative stimulating energy can be adjusted or selected to enhance effectiveness in alleviating the symptom, typically by increasing voltage, current, and/or power, by adjusting the duration of the duty-cycle, the shape of the pulse, or the like.

The methods for restoring airflow according to the present invention will typically further comprise recording data and correlating the data with the ability or inability of a particular level and type of stimulating energy to alleviate or terminate the snoring or apneic event. As this data is collected over time, a baseline for treating individual patients can be generated on a patient-by-patient basis. Thus, for individual patients, the detection of those patient systems and ambient conditions which are likely to result in an apneic or snoring event can be more accurately detected as the data are collected and the baseline refined over successive uses. Thus, as patient treatment continues over time, the ability to accurately detect the onset of a snoring or apneic event will improve. These methods may further comprise storing baseline data locally on the treatment device, on a remote device which is carried by the patient and/or on further remote devices at central locations.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a block diagram of a diagnostic device attached to a patient.

FIG. 1B shows how incoming parameters are collected and delivered through a bus to a microprocessor which drives a pulse generator and a pair of stimulatory electrodes in a therapeutic device.

FIG. 1C illustrates a necklace adapted to carry components of the systems of the present invention.

FIG. 2 is a flowchart illustrating the collection of breathing sounds from a patient's neck using a surface microphone.

FIG. 3 is a flowchart illustrating the use of electrical muscle stimulating to contract a genioglossus muscle to open an airway to restore normal breathing.

FIG. 4 is a block diagram of a diagnostic device used in combination with a remote device running application software.

FIG. 5 illustrates the hardware of a diagnostic device in great detail.

FIG. 6 illustrates the hardware of a therapeutic device in great detail.

FIG. 7 illustrates exemplary locations for placement of different patient sensors on the patient's body.

FIG. 8 illustrates the layout of the microprocessor used for collecting data from a plurality of sensors.

FIG. 9 illustrates the collection of noise data from sensors.

FIG. 10 shows exemplary filtering of environmental noise.

FIG. 11 illustrates the serialization of data collected from the sensors.

FIG. 12 is a block diagram showing the transmission of data between the microprocessor and memory as well as between remote devices.

FIG. 13 illustrates additional sensor placement locations.

FIG. 14 illustrates local and remote detection of data in a therapeutic device.

FIG. 15 illustrates disposable and non-disposable components of the therapeutic device.

FIG. 16 illustrates the information flow to an external device to process sleep diagnostic data collected by device sensors.

FIG. 17 illustrates information processing in a therapeutic device using both an external device and a processor imbedded in the therapeutic device.

FIG. 18 illustrates processing of filtered data to detect apneic or snoring events.

FIG. 19 is an acquired wave form generated by the analysis of FIG. 18.

FIG. 20 illustrates four steps in apnea detection.

FIG. 21 illustrates the genioglossus muscle under the tongue.

FIG. 22 illustrates measurement of electric current.

FIGS. 23A-23D illustrate exemplary wave forms generated by a therapeutic device.

FIG. 24 illustrates a training cycle for calibrating a therapeutic device.

FIG. 25 further illustrates the steps involved in a training cycle.

FIG. 26 is a graph illustrating the average or mean value of the sensor data reference values in a given duration period.

FIG. 27 is a block diagram illustrating the relationship between an event detection thread, a stimulation thread, and a stimulation monitoring thread.

FIG. 28 illustrates how encrypted data are maintained and uploaded to remote storage (in the “cloud”).

### DETAILED DESCRIPTION OF THE INVENTION

The following terms and phases used in the specification and claims are defined as follows:

“Airflow” means airflowing through a patient's nose and mouth, between the soft palate and rear of the tongue, through the trachea into the lungs.

“Airflow disruption” means snoring or other apneic events which occur as the soft palate or tongue deform and interfere with airflow, typically during sleep.

“Airflow restoration” means the cessation of an airflow disruption by stimulation of muscles of the upper air way which open the air way to end the snoring or apneic event.

“Ambient condition” means temperature, humidity, light intensity, noise level, and other environmental conditions in which the patient is sleeping.

“Apneic event” refers to stopped breathing, disruption on heart rhythm, and other conditions experienced by a patient as result of sleep apnea.

“Arousal threshold” means an intensity of muscle stimulation which has or will wake or otherwise arouse a sleeping patient.

“Baseline” means (1) data collected for an individual patient which correlates the likelihood that the patient will suffer snoring and/or an apneic event with symptoms and ambient conditions experienced by the patient in real time and (2) data collected for an individual patient that correlates an intensity or other property of stimulation which has been effective in reducing snoring and/or an apneic event with symptoms and ambient conditions experienced by the patient in real time.

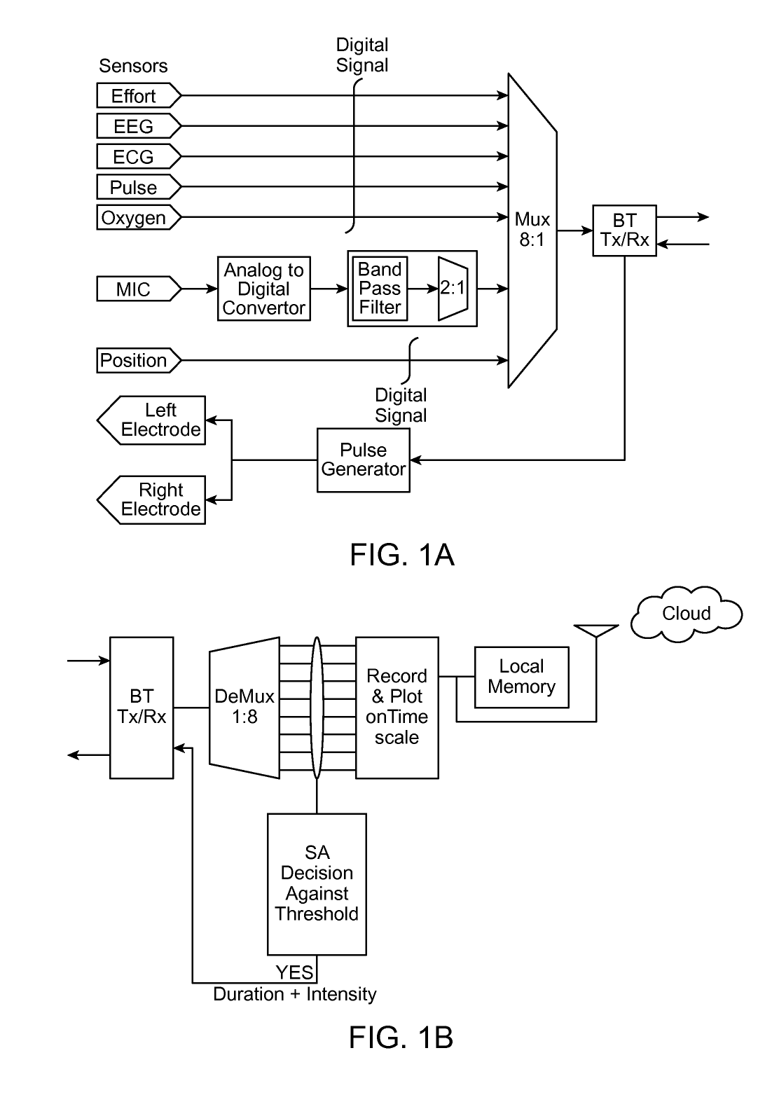

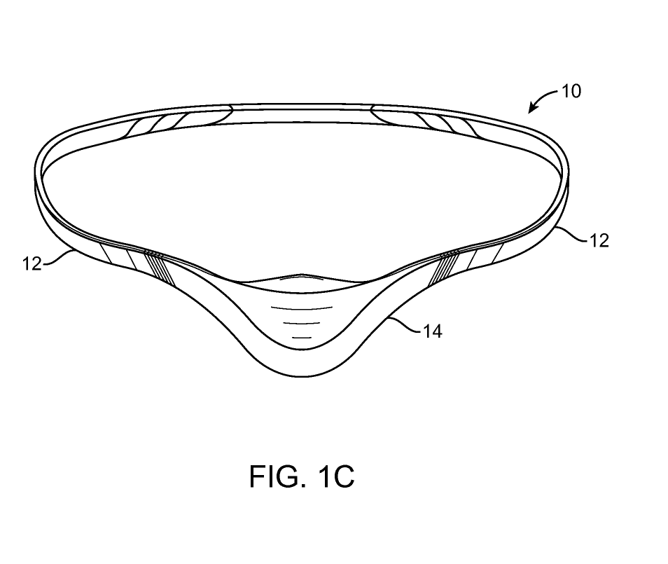

“Component worn by the patient” means a collar, band, strap, hat, vest, visor, necklace, or other platform wearable on or near the patient's neck which carries a microprocessor, memory, power source and optionally sensor(s) which form portions of the devices of the present invention. An exemplary necklace is illustrated in FIG. 1C.

“Electrical delivery element” means an electrode, pad, gel pad, cuff, or other device intended for placement against a patient's skin to deliver an Stimulating Energy or another electrical signal transcutaneously to a muscle or elsewhere. Electrical deliver elements may be formed integrally with a component worn by the patient or may be formed separately from the component.

“Stimulation” means causing a muscle of the upper airway to contract in order op open the patient's air way.

“Stimulating energy” means energy being delivered to and/or through a patient's skin to stimulate the muscle of the upper air way. Stimulating energy will usually be electrical and will be characterized by current, power, voltage, frequency, pulse width, pulse repetition rate, duty cycle, and waveform.

“Transmit” refers to the wireless and/or wired delivery and exchange of digital and analog information, where wireless transmission is typically achieved by radio, Bluetooth®, and WiFi, and wired transmission is typically achieved by connecting wires or cables or by conductors formed on solid state devices.

Structure and Operation of a Therapeutic Embodiment of the Device (from 61/808,937)

In one embodiment, detection and treatment of sleep apnea uses a device in shape of collar band and a software application running on an external device, such as a hand held device or any mobile computing device. This collar device is anchored around patient's neck using removable adhesive pads. The device works in conjunction with separate sensors placed on an ear lobe and the chest. Sensors on the collar device may include a microphone to capture tracheal sounds, a position sensor, such as an accelerometer, to detect patient sleep position, a pulse sensor, a snore detection microphone, and the like. Sensors on the chest may include a breathing effort sensor, such as gyroscope, that detects the chest expansion related to breathing, and one or more electrocardiogram (ECG) electrodes for detection of cardiac arrhythmias. Collar device also have s two electrodes (number of electrodes can be variable) used to transmit electrical current stimulation to the patient. Sleep apnea is detected and treated continuously. The sensors collect the airflow information, heart rate, breathing effort, heart activity, muscle tone (EMG), blood oxygen level (SaO2), and body position. The collar device collects and digitizes data from all sensors. The sensor data are processed internally by the microprocessor to detect the apneic event, These data are typically also sent wirelessly to the hand held device.

FIG. 1A shows a block diagram of the device and sensors attached to patient. Software application running on the mobile device or any computing device process and evaluate the data to identify the sleep apnea episodes. A measured response is transmitted to the patient resulting from intensity of each episode as well as the impedance of the stimulation current path. Airflow is primary parameter and measured by the non-invasive technique. Airflow is measured by capturing the sound-created by air passing through the windpipe or trachea. Sound frequency varies with the volume of air flowing through the trachea. Variation in airflow is correlated to effort, blood oxygen level, pulse heart rate and several other vital parameter. Sleep apnea is detected when measured parameter are compared against the individualized threshold. The system may be passed through a training cycle where the parameters are initialized and individualized thresholds are established. Conversion of tracheal sound frequency to airflow is also dependent on the body positions. When the subject/patient changes position a new threshold may be established for that position.

FIG. 1B shows how incoming parameters are collected and delivered through a bus to the microprocessor that drives a pulse generator and a pair of stimulatory electrodes. For example, the device may collect data from eight sensor channels:
1. Airflow
2. Oxygen saturation
3. Heart rate
4. Breathing effort
5. Body position
6. Snoring
7. ECG
8. EMG.

Structure and Operation of a Diagnostic Embodiment of the Device.

Breathing sound is detected from the patient's neck using a surface microphone. An exemplary collar 10 having a pair of wings 12 for temporary placement around a patient's neck includes a module or pendant 14 which will lie over a lower center region of the neck when the collar is worn. The collar is open at the back allowing a a patient to conveniently don and remove the collar using only a single hand. The device electronics, as described elsewhere, may be incorporated into the module 14 for both the diagnostics and therapeutic devices. In particular, the module 14 will typically have one or more microphones for detecting bearthing and ambient sounds.

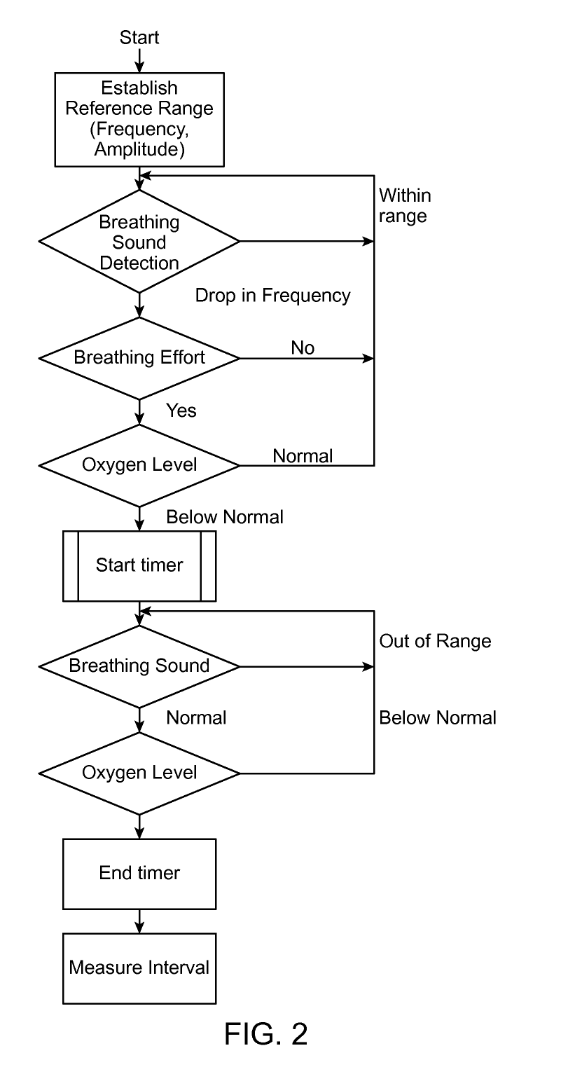

An analog signal from a surface microphone on the module detects breathing sounds and the signal from the surface microphone is amplified and digitized. The digitized signal contains information about the amplitude and time period of the breathing sound. These values could be unique for a patient based on the patient's physiology. Normal breathing for a limited time establishes a normal range for the amplitude and time period. This range is used as a reference, as shown in FIG. 2. During sleep, additional channels of physiologic information may be collected. The variation in the breathing sound frequency and amplitude is observed in conjunction with oxygen saturation level, breathing effort, heart rate and electrocardiogram signal. A change in the frequency of the tracheal sound from reference range triggers the start of decision loop. If the effort parameter is positive, the comparison loops forward to the next step. In second step blood oxygen saturation level is detected. A change in oxygen level below normal indicates the sleep apnea event.

Structure and Operation of Another Therapeutic Embodiment of the Device.

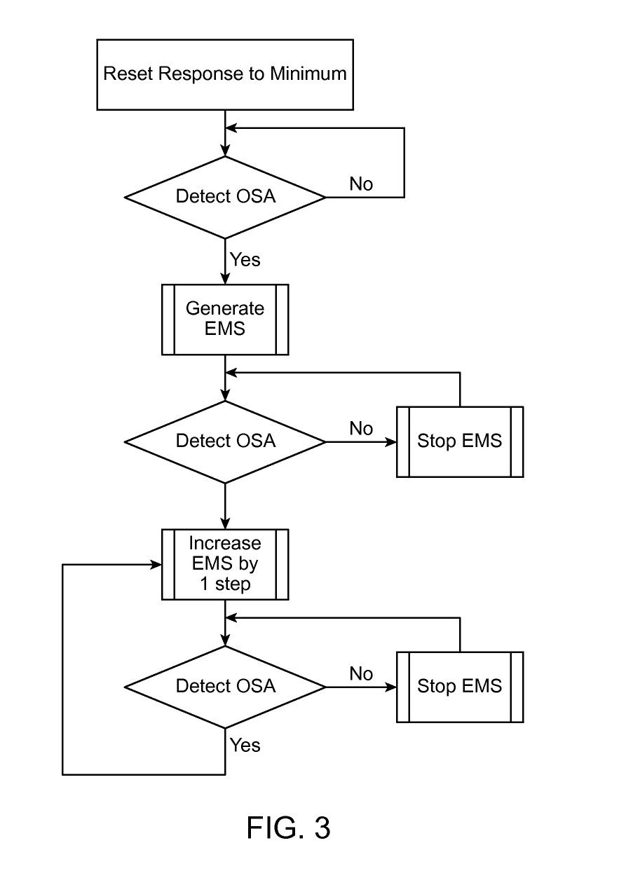

FIG. 3 is a flowchart illustrating the use of electrical muscle stimulation (EMS) to contract a genioglossus muscle. EMS is applied during the obstructive sleep apnea (OSA) event to the genioglossus muscle to contract the muscle and open the airway enough to restore normal breathing. The magnitude of stimulation is a calibrated and customized for the individual and conditions. Prior to EMS application, an obstructive sleep apnea event may be detected using a plurality of physiological parameters collected from the patient. An analysis of al parameters establishes the APNEIC/SNORING event.

The EMS response is initially reset to a minimum estimated or calculated to be the minimum required in order to restore normal breathing. Response is increased in steps if obstruction in airway persists. EMS is transmitted to patient via a pair of gel pads or other electrical delivery elements that makes contact with skin beneath the genioglossus muscle. The stimulation level is kept below arousal threshold level (typically a stimulation current level at which subject starts waking up). A change in condition (body and/or neck position) or a new detected impedance value will typically reset the response level to the previously set minimum. The duration and frequency of the response is typically dependent on the duration and frequency of the detected APNEIC/SNORING event. The algorithm works in a loop as shown in the FIG. 3, increasing and decreasing the EMS intensity during treatment.

Structure and Operation of Another Diagnostic Embodiment of the Device.

Airflow is measured from a neck surface using an acoustic signal. A microphone is attached to the neck surface, and a real time sound signal is collected. After powering up, a variation in the signal amplitude is recorded in real time. The device buffers the signal in the memory. The recording interval is adjustable based on the duration and number of interruptions detected. An interruption is detected as a change in the cycle time and amplitude due to obstruction or lack of breathing effort. Two parameters, amplitude and breath cycle, from the detected sample period are tracked. The amplitude and breath cycle is averaged over the sample for a predefined time period. The predefined time period is a moving average updated by adding the new detected signals and deleting the oldest sample data. Thus, the average respiratory cycle tracks the change in breathing during different stages of sleep.

The position of the patient being tested can be used to initiate sampling. When a change in the patient's sleep position is detected, the buffer sample can be initialized and new averages be detected or calculated. A latest value of the averaged amplitude and respiratory cycle time for each position is kept in the buffer. When the patient returns to a previously recorded position, the previously calculated average is used as the initial value and until a new complete sample period is recorded.

Structure and Operation of Another Therapeutic Embodiment of the Device (from 61/827,744)

EMS response patterns are generated using electrical pulse generator and gel pads with wire mesh. A pulse generator generates two or more pulses of variable intensity. These are transferred to wire grid/meshes on the gel pads. Gel pads make contact to the throat muscles.

A pseudo-random pattern generator sends a digital signal to the pulse generator, which is converted into two or more pulses of variable intensity, duty cycle and duration. Each pulse's intensity varies with time during the asserted interval. These pulses are transferred via the mesh or wire grid to contact points on the skin. The wire grid/mesh spreads the electric pulse in horizontal and/or vertical fields across the skin in contact with gel pads. Two or more pulses spreading across the gel in different directions with different intensity creates a unique EMS pattern.

EMS patterns generated as described above are applied to throat muscles via gel pads making contact to the neck surface. Pseudo-random pattern signal generator cycles through the different codes and each code corresponds and translated into a unique pattern on the gel pad.

A response to the stimulation patterns is detected via real time by the airflow monitor. The codes which maximum the airflow restoration are stored as an optimum response for the patient under the particular patient and ambient conditions recorded. These correlations can be stored locally or remotely used as part of self-learning process (calibration and adoption) for each patient. Any change in the condition will restart the cycle of different stimulation patterns until the best response is detected. The algorithm works in a loop with the airflow detection mechanism and response codes corresponding to every condition are stored and recalled as airflow obstruction is detected under those conditions.

Structure and Operation of a Snoring Therapeutic Embodiment of the Device (from 61/27,745)

EMS response patterns are generated using electrical pulse generator and gel pads with wire mesh as described above.

A response to the stimulation patterns is detected via real time by a snoring monitor. The codes which maximum the airflow restoration are stored as an optimum response for the patient under the particular patient and ambient conditions recorded. These correlations can be stored locally or remotely used as part of self learning process (calibration and adoption) for each patient. Any change in the condition will restart the cycle of different stimulation patterns until the best response is detected. The algorithm works in a loop with the airflow detection mechanism and response codes corresponding to every condition are stored and recalled as airflow obstruction is detected under those conditions.

### System Descriptions

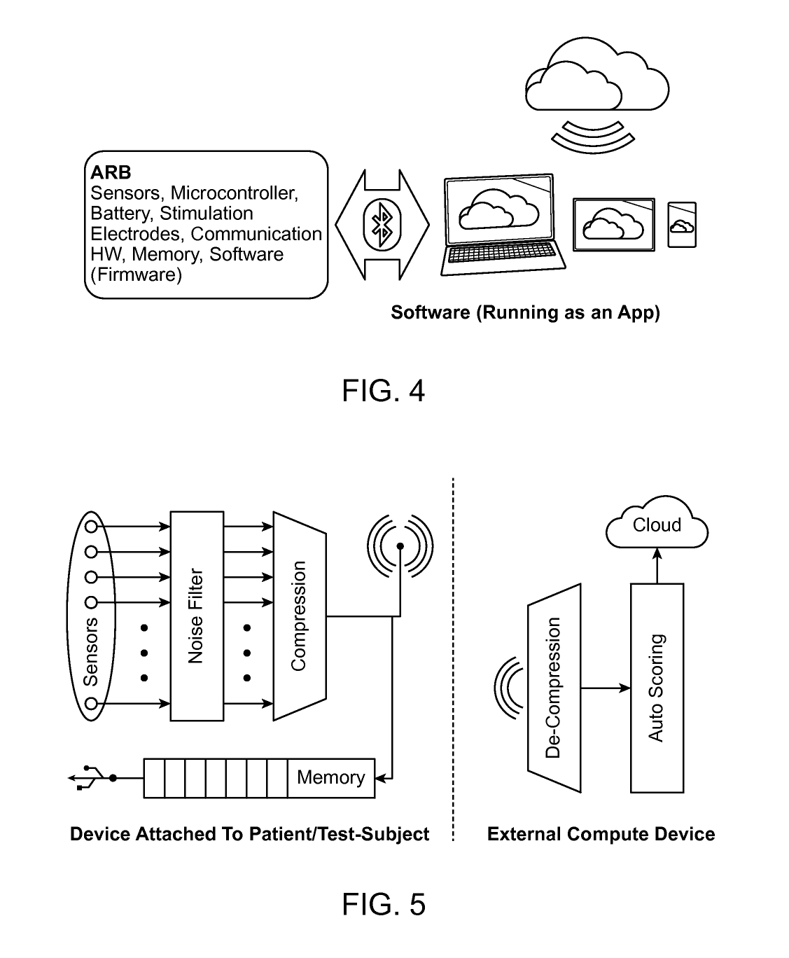

The wearable diagnostic and therapeutic devices of the present invention are designed to operate independently as well as in conjunction with the external computing devices, such as a smart phone, a personal digital assistant (PDA), a tablet computer, a personal computer, or a specially designed mobile or table top controller which can communicate with the wearable component described herein. The wearable component has built in memory, power, and a microcomputer or microcontroller that can operate independently. When device is working in conjunction with an external device, it has extended memory and better computing capability. Optionally, the external device can also upload the sleep data to a remote storage facility or the “cloud.” (See FIG. 4).

Both diagnostic and therapeutic versions of the devices of the present invention have common features. Both will have sensors to detect patient and optionally ambient conditions. The diagnostic devices may have more sensors to make better diagnoses and to provide a number of channels (sleep parameters) required for common sleep diagnostic protocols. The therapeutic devices need only basic sleep sensors but require stimulation hardware that is unnecessary on diagnostic devices. More complete descriptions of both devices are given in following sections.

The diagnostic devices are passive devices that collect and store sleep data but do not provide therapy to the patient. Software processes the collected sleep data real time and can automatically evaluate the data and generates a “sleep score.” Raw sleep data can be locally stored, and can later be transferred to the external device (e.g. a smart phone) where the data may be processed and uploaded to cloud if desired. (See FIG. 5).

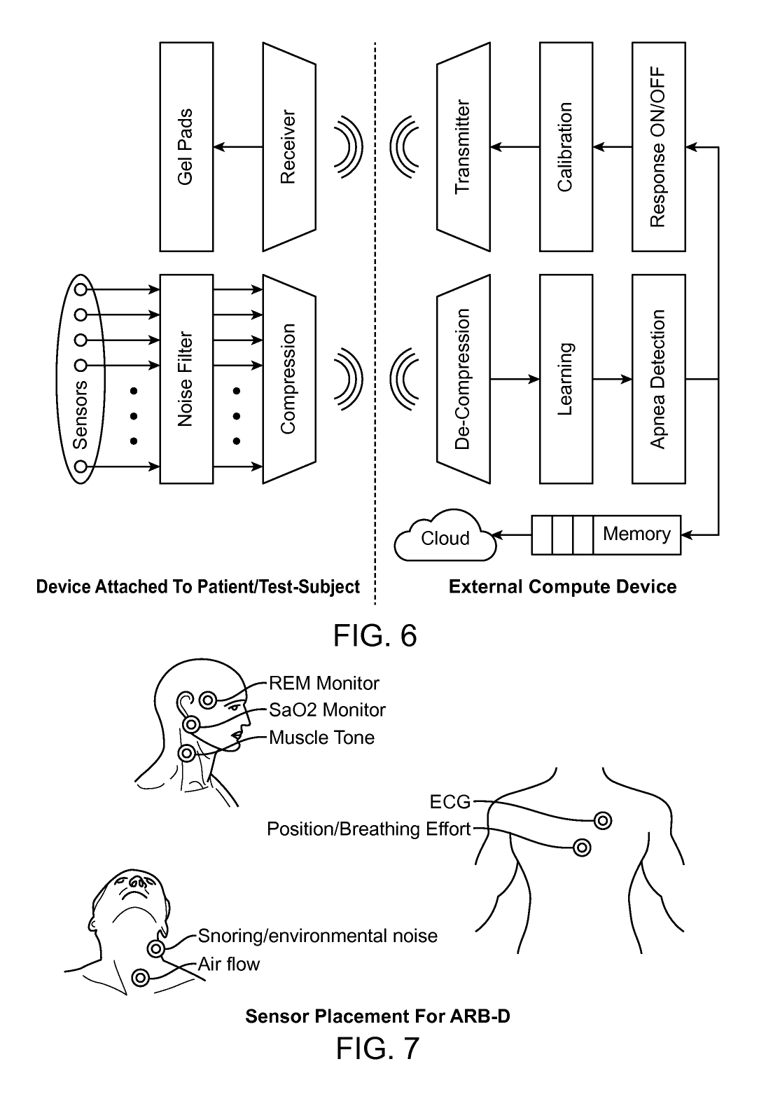

The therapeutic devices are active devices that both detect sleep apnea or snoring and generate a therapeutic response in form of electrical or other energy pulses. The pulses are delivered to an upper air way muscle typically via gel pads or other electrical delivery elements attached to the skin on the throat under the chin. The therapeutic devices will usually carry a subset of sensors carried by the diagnostic device. The therapeutic sensors will collect sufficient sleep data to identify the onset of apneic event, snoring episode, or obstruction in the airflow. Upon identification of the onset, the therapeutic device generates a series of electrical pulses that are transmitted to patient to stimulate the target muscle(s). As soon as the normal airflow is detected, the pulses are stopped. The therapeutic device records data relating to the apneic/snoring episode. Optionally, the therapeutic device and/or the associated external device will have self-learning capability to adjust stimulation levels and/or timing to the individual patient and the ambient. Self-learning or device calibration and adoption is typically accomplished by a software program running on the external device that is connected to the wearable device or optionally another interface device worn by the patient. (See FIG. 6).

The Diagnostic Device

The diagnostic device consists of sensors, a microprocessor or controller, local memory storage, and battery or other power source. Some of the sensors are embedded in the device main assembly and others may be connected via leads or have a wireless connection to the main assembly unit worn by the patient. The diagnostic device may include some or all of the following sensors:

Surface microphone for tracheal sounds

Microphone to capture the snoring sound

Microphone to capture ambient and other non-breathing sounds

Pulse oximeter to measure blood oxygen saturation level

Gyroscope for body position and motion detection

Gyroscope for the breathing effort detection

Electrocardiogram (ECG) sensor

Sleep stage sensor

Muscle tone sensor

The surface microphone to capture tracheal sound will be place on the neck over trachea and below the notch (thyroid cartilage) facing towards the body to capture the breathing sound (See FIG. 7).

Arrays of microphones may be placed on the both sides of neck facing outwardly to capture the snoring and environmental noises. A SaO2 sensor, such as a pulse oximeter, is placed either on earlobe. A surface SaO2 monitor can also be used by placing on a side of neck. The gyroscope/motion detectors monitor breathing effort and/or body position and are placed on the chest. The ECG sensors (typically electrodes) are placed on the chest above heart to monitor heart activity. Sleep stage is monitored by rapid eye movement (REM) detectors placed on the side of either eye, and a muscle tone sensor is placed on the neck muscles. Other sensors might include skin resistivity sensors which relate to tissue hydration and impedance.

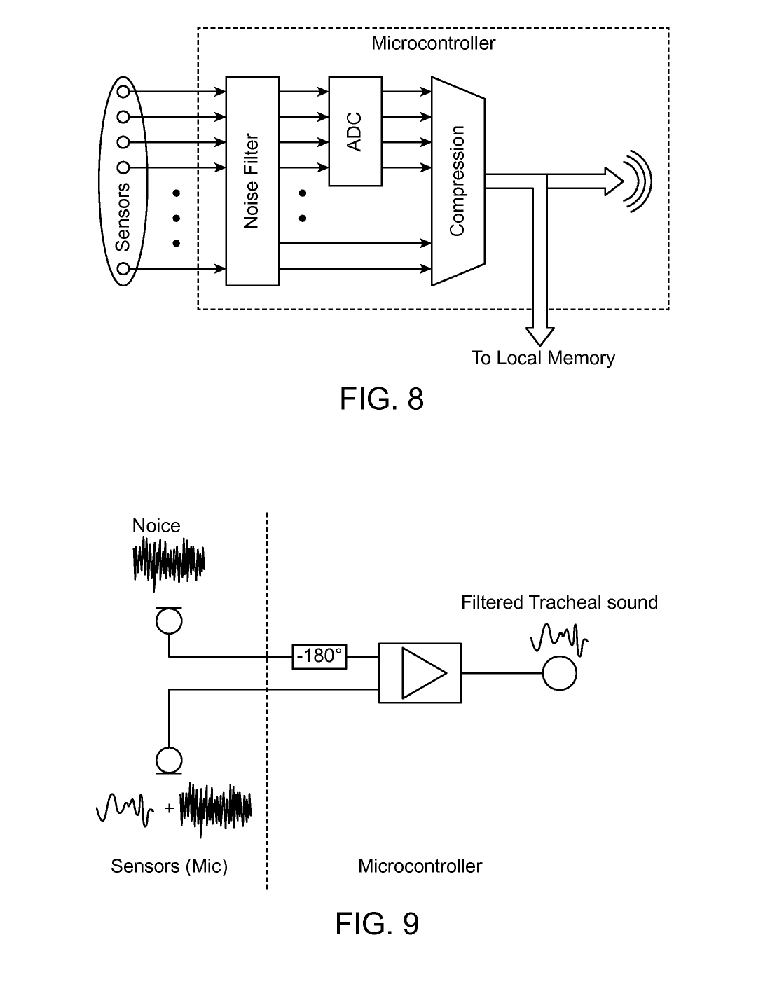

The microcontroller in the diagnostic device performs several important functions. It processes the sensor data, packages the processed data, and wirelessly transmits the data packets to the external device. (See FIG. 8).

The real time sensor data is cleaned by the microprocessor prior to transmission. The sound data associated with tracheal airflow sound signal is cleaned and all environmental noise is filtered by active noise cancellation performed by the microprocessor. (See FIG. 9).

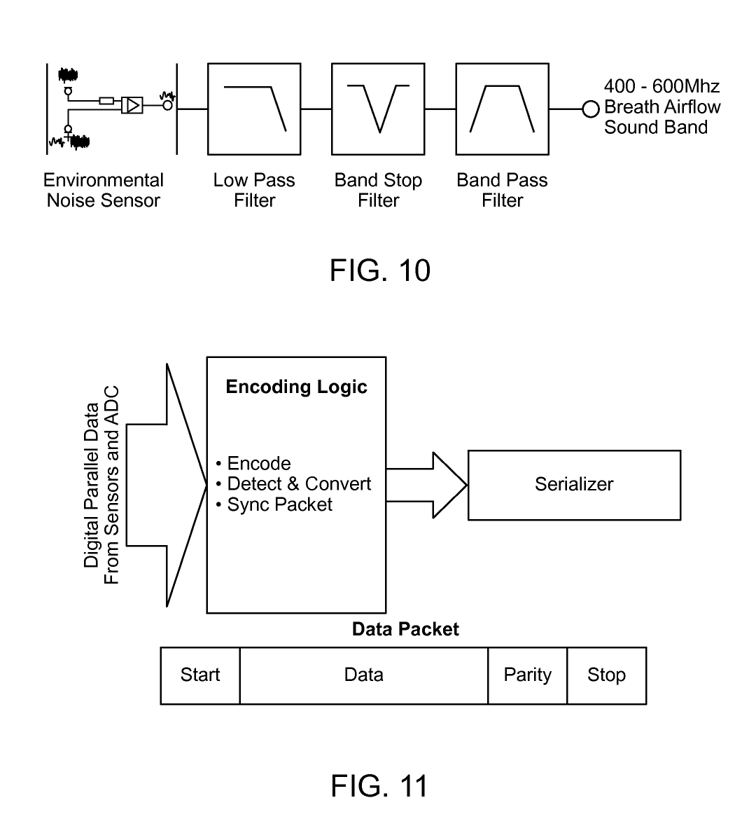

The filtered tracheal sound signals still have frequency components not associated with the respiratory airflow in the trachea. The frequencies associated with respiratory airflow are in the range of 400-600 MHz. The filtered signal is passed through a band-pass filter to extract the respiratory airflow frequency band. Sound signal from the tracheal microphone is passed through an active noise cancellation (ANC) block described to eliminate the environmental noise from the tracheal sound. The tracheal sound microphone also captures the high frequencies sounds associated with neck movement. The signal is passed through low pass filter (LPF) to remove these movement related sounds. In next steps signals is passed through the combination of a band stop filter and a band pass filter to extract the target 400 MHz to 600 MHz signal associated with breathing. (See FIG. 10).

For analog sensor data, the microprocessor converts the filtered data (mostly representing acoustic sound) to digital. The sensors providing digital data don't require filtering. The digital signals are encoded into one data packet by the microprocessor and then transmitted to the external device. (See FIG. 11).

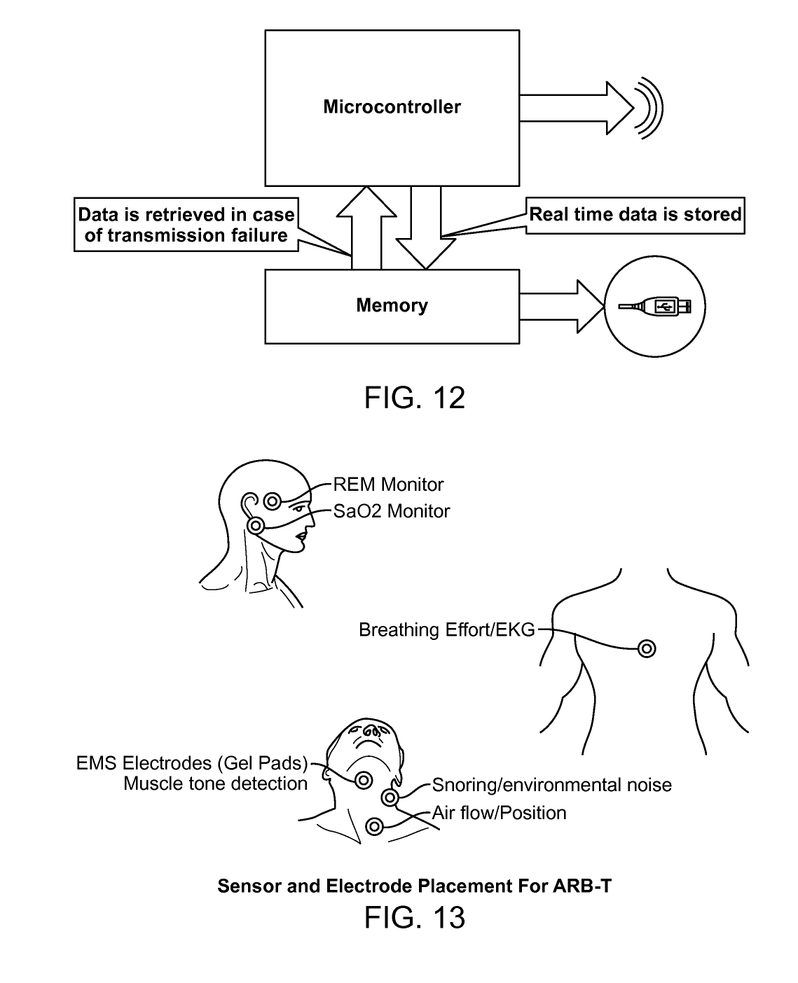

The diagnostic device memory stores the sleep data, typically as a backup in case connection to primary memory in the external device is broken. When connection is restored, the data can be transmitted. The storage capacity will typically be limited but should be sufficient to hold data from a full test duration. The memory is usually flash storage so that data are not lost when device is powered off or out of battery. Data can be downloaded from external device via external USB or other port to a computer or other location. (See FIG. 12).

The battery may be a rechargeable Lithium Ion battery to power to all components and circuitry of the wearable device. The battery will also provide the power to some or all sensors. The battery should have enough capacity to provide power for an entire test with a full charge.

The Therapeutic Device

The therapeutic device includes the sensors, microprocessor, memory, battery, and electrical delivery elements (electrodes) for EMS. The therapeutic device has sufficient sensors to detect airflow obstruction and/or an apneic event. It will usually also include a body position sensor, SaO2 sensor, and sleep stage detector. The main purpose of sensors is to identify an airflow obstruction or an apneic event, not to provide a full diagnosis. Thus, only a subset of the diagnostic sensors are useful, including:
Surface microphone for tracheal sounds
Microphone to capture the snoring sound
Microphone to capture ambient and other non-breathing related noises
Pulse oximeter to measure blood oxygen saturation level
Gyroscope for body position and motion detection
Gyroscope for the breathing effort detection
Sleep stage sensor
Muscle Tone detection and picture
(FIG. 13)

The microprocessor processes the sensor data and transmits to the external device as with the diagnostic device. The microprocessor in the therapeutic device, however, may also provide additional logic to further process the sensor data to identify beginning of a snoring or apneic event locally. Once onset of the snoring/apneic event is detected, either locally or by the external device, the microprocessor generates an EMS stimulation signal, typically a series of pulse. The microprocessor turns of the stimulation as soon as the sensor data indicates the snoring/apneic event has subsided. The microprocessor will also increase or otherwise adjust the stimulation signal if the snoring/apneic event does not subside within an expected time period.

The therapeutic device detects the start of the snoring/apneic event or the obstruction to airflow. Sensor data is processed against the preset criteria to determine if this is normal airflow or obstructed airway. There are two ways may be used:

Local Detection: Digitized sensor data is processed by signal processing logic in the microprocessor. Firmware of the device defines an initial baseline criteria. As device is used, built-in logic updates the criteria based on data collected regarding both ambient and patient conditions.

Remote Detection: Similarly to the diagnostic device, the sensor data are packaged and sent to the external device. This external device has software which processes the data after decoding it. As soon as the onset of an airflow obstruction or other apneic/snoring event is detected, the external device instructs the hardware attached to patient/user to treat the event. The response is terminated as soon as normal airflow is detected (See FIG. 14).

The therapeutic device is configured to initially generate a default stimulation based on the user/patient profile. The hardware is configured by the firmware that can be updated. As the device is used it self calibrates based on the collection of data representing the patient's response to particular stimulation under different patient conditions. In a particular course of treatment, e.g. over one night, as patient and ambient conditions change, the device keeps adjusting the stimulation response to optimize the results. The calibration and learning process is performed by the built in logic in microcontroller.

Once an obstruction in the airflow is detected, an EMS response is turned ON. The EMS response is in typically the form of electric pulses, and the wearable device hardware generates a measured and customized response. The EMS pulse has following characteristics:
Duration
Duty cycle
Amplitude (intensity)
Slew rate
Pattern

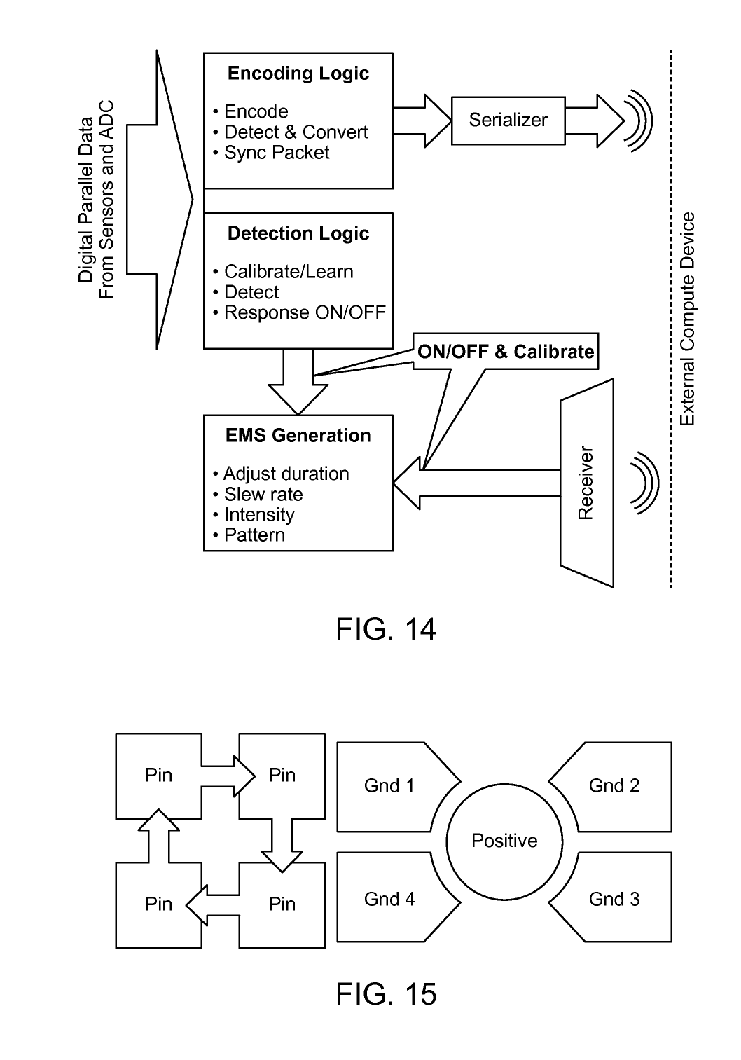

The hardware components of the therapeutic device may be partially disposable. The therapeutic device may have fixed metal electrodes that are covered with the disposable pads. These pads attach to the patient skin under the chin. EMS is transferred to throat muscles via these pads. To create different stimulation patterns, the gel pads are divided into different regions. Sending pulse or enabling ground in those regions generates different patterns or current paths through muscle. A knob may be used to cycle through different pattern to select the one with best response. (See FIG. 15).

Both the diagnostic and therapeutic devices use software based digital signal processing of the sensor data. Detection of the snoring/apneic events are also relies on software based digital signal processing. Calibrations and adjustments of the sensor output and EMS response during sleep test or therapeutic use is also done by software. Software also used for the data management and record keeping for physician and compliance tracking.

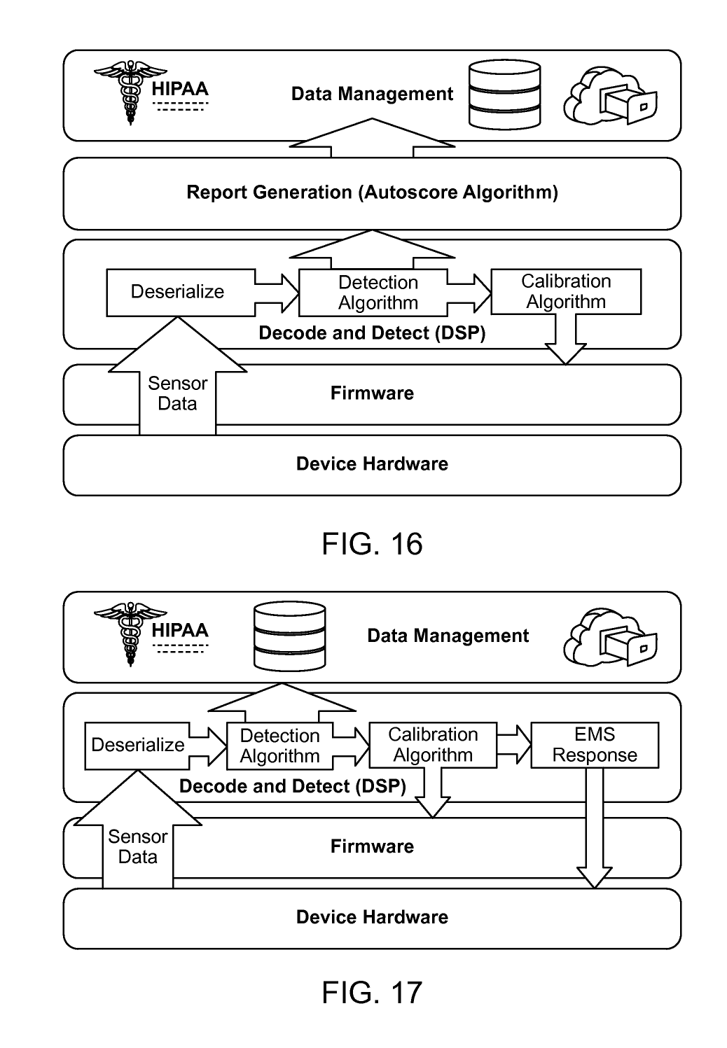

The diagnostic device uses the external device to process the sleep diagnostic collected by the device sensors. A software digital signal processing (DSP) algorithm does the signal processing of the digitized data. Software determines the apnea hypopnea index (AHI) for the test subject then scores the processed output. (See FIG. 16).

Firmware is the software that configures the hardware of the device for operation. This software is uploaded to the hardware and can be updated periodically. Firmware provides the initial values for the sensor data, these values are calibrated and adjusted as the device is used and firmware gets updated during the process. In this component of the diagnostic software serialized data is decoded or de-packetized generating the individual sensor data at the same time scale. Signal processing algorithms take the sensor data and identify changes in the respiratory airflow.

This component also calibrates the sensors based on received data if there is certain changes (for example, body position) are detected. The calibration is done by updates sent to firmware.

The diagnostic device generates the sleep test results and provides the AHI index for the collected sleep data. The auto score algorithm separates the airflow obstruction events in categories of apnea (complete airflow obstruction) and hypopnea (partial airflow obstruction).

Sleep test report is encrypted and uploaded to the cloud according to data security standards required by the HIPAA. The data is made available to the prescribing physician. Report is also tested for the validity of the data before upload.

The therapeutic device uses both external device and embedded processor to process the collected sleep data. The software digital signal processing (DSP) algorithm does the signal processing of the digitized data on the external device. In parallel the firmware controlled algorithm running on the embedded processor does the processing independently. Both are used to identify the start of apneic/snoring event. EMS response is triggered ON and turned OFF by the software as start and end of apneic/snoring event are identified. (See FIG. 17).

Firmware is the software that configures the hardware of the device for operation. This software is uploaded to the hardware and can be updated periodically. Firmware provides the initial values for the sensor data and EMS response values, these values are calibrated and adjusted as the device is used and firmware gets updated during the process.

The therapeutic device software may use serialized data which is decoded or de-packetized, generating individual sensor data at the same time scale. Signal processing algorithms take the sensor data and identify changes in the respiratory airflow. The software may also calibrate the sensors based on received data if certain changes (for example, body position) are detected. The calibration is done by updates sent to firmware. Calibration of the EMS signal is also done based on the sensor feedback. The EMS signal is turned ON at the onset of the apneic/snoring event and turned OFF as soon as the apneic/snoring event is over.

The therapeutic device generates a usage report during each use. This report is usually encrypted and uploaded to the cloud according to data security standards required by the HIPAA. The data are made available to the prescribing physician. The validity of the data is usually tested before upload of the report. This report can provide compliance tracking for healthcare payers.

### System Integration and Operation in Detail

The therapeutic device for the treatment of sleep apnea and snoring includes three components for the detection of an apnea event, the calibration of sensors and electrical stimulators, and generation of an EMS signal, respectively. The EMS signal is sent to the patient's genioglossus muscle or other muscle of the upper airways. The detection of the apneic/snoring event and the stimulation of the genioglossus muscles occur in real time i.e. as soon as an apneic/snoring event is detected. Although the diagnostic device is intended primarily for in-home-diagnosis of OSA, data may be sent to HIPPA-compliant external storage for further analysis by sleep apnea specialists.

The detection algorithm acquires data from the tracheal sound microphones, the body position and motion detection gyroscope(s), the SaO2 sensor, the breathing effort monitor (gyroscope), the muscle tone sensor and the sleep stage sensors (REM sleep detector and/or EEG) to detect all apnea and hypopnea events. The microphones that collect the snoring data and the data from the external environment are used to cancel out any non-breathing related noise from the tracheal sound microphone, using noise-cancellation techniques. To enhance the accuracy of the detection, several parameters, including blood-oxygen saturation levels, the sleep stage, muscle tone, ECG readings and breathing effort are measured in conjunction with the tracheal sound. Due to differences in patient body masses, the sound of a patient's breathing at different body positions, and the conductivity of the patient's heart, the detection algorithm adapts to the above patient parameters.

Due to the high variability of an individual's body signals associated with the sleep parameters while asleep, it is essential to recalibrate all sensors and detection thresholds to take into consideration the different body positions, sleep stages and breathing rates. These calibrated parameters will then alter the parameters used to detect the changes in airflow, blood-flow saturation, and muscle tone, and subsequently determine the current of the EMS signal. So for each detected Sleep Apnea episode a calibrated and measured EMS response is sent to the muscle.

Once the change in the respiratory airflow has reached a certain threshold, the firmware turns on the EMS Signal, which will then stimulate the genioglossus muscle. This signal indicates the beginning of an apneic/snoring event. The voltage of the electrical stimulus depends upon the constantly monitored impedance of the patient's skin, electrode contact and the muscle. Patient's total percentage of fat under the skin, body position, skin moisture level and muscle tone strength determines the intensity of electrical stimulation. This adaptive signal will then prevent the patient from undergoing an apnea event. Response is terminated as soon as normal breathing is restored.

Both the diagnostic device and the therapeutic device are designed to work in combination with the external device. The hardware of each patient wearable device, however, is designed to function without the external device in case of loss of connection between wearable device hardware and the external device.

All sensors in the device hardware work independently and are integrated vertically. The sensor data noise filtration is also integrated vertically. The data decoding and packetizing is common. Similarly the data transfer and receive between the hardware device and external device is also common for all sensors. The therapeutic device has a redundant local data processing function embedded in the device hardware. It can work independently as well as in combination with the external device running data processing.

In the diagnostic device, respiratory airflow is detected using tracheal sounds. Any disruption in the airflow is detected and correlated with the other sensor data like SaO2 data and sleep stage data. The sensor outputs are then processed and events identified by the self-scoring algorithm and report uploaded to the cloud. Following are details:

The following defines the technique used to acquire and process sound waves from a patient suffering from Obstructive Sleep Apnea (OSA). In this procedure, a microphone is attached to the anterior portion of the patient's neck, directly above the trachea. This microphone captures the tracheal sounds associated with the respiratory airflow. Once the patient has entered REM sleep, the software begins to acquire tracheal sound data from the microphone.

In order to analyze the data, it is first cleaned of the environmental noises by the frequency filters. Another microphone or set of microphones is used to capture the environmental noises (non-tracheal sounds). All frequency components from non-tracheal microphone that fall in the tracheal sound frequency spectrum are subtracted from tracheal microphone sound data. It is then converted from an analog signal to a digital signal. In software DSP, every 0.2 seconds the raw data must undergo a processing algorithm. This algorithm applies the Fast Fourier Transform to the raw tracheal sound data and generates the respective power spectrum for that data. Over the course of the night, the data is summed to generate a plot of the Power Spectral Sum.

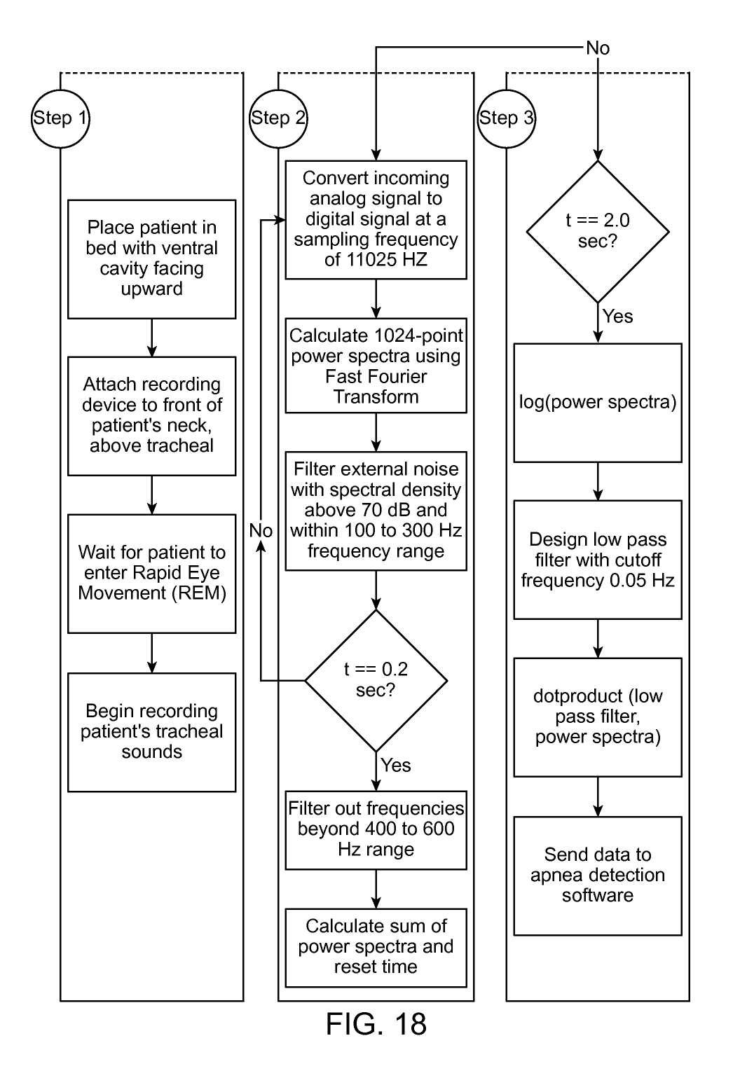

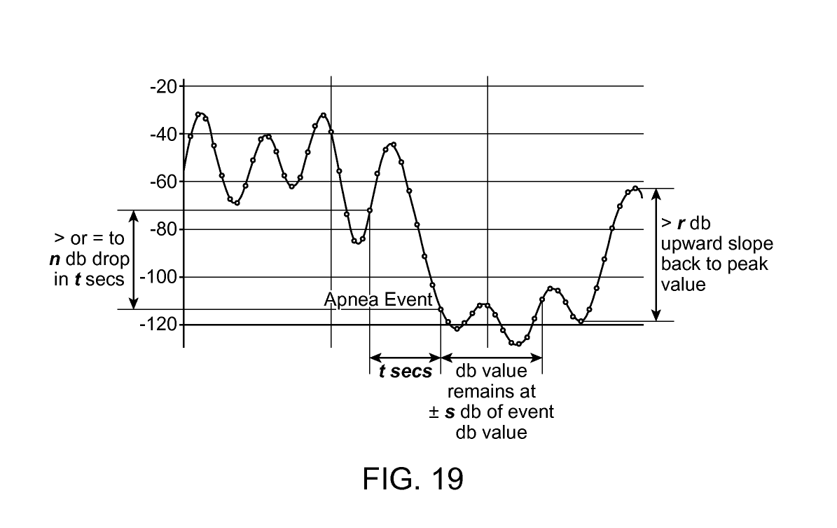

As this process is occurring, all frequencies outside of the range of 400 to 600 Hz are filtered out of the acquired data. Every 2 seconds, a logarithmic moving average of the data is generated for the purpose of filtering out any frequencies beyond the interval −0.05 Hz≦ω≦0.05 Hz. This process smooths the data for apnea/hypopnea detection. After 2 seconds, this data is passed into C# code for further analysis and apnea/hypopnea detection. (See FIG. 18). The acquired waveform looks like as in FIG. 19. The software DSP processes this waveform and identifies the apnea events.

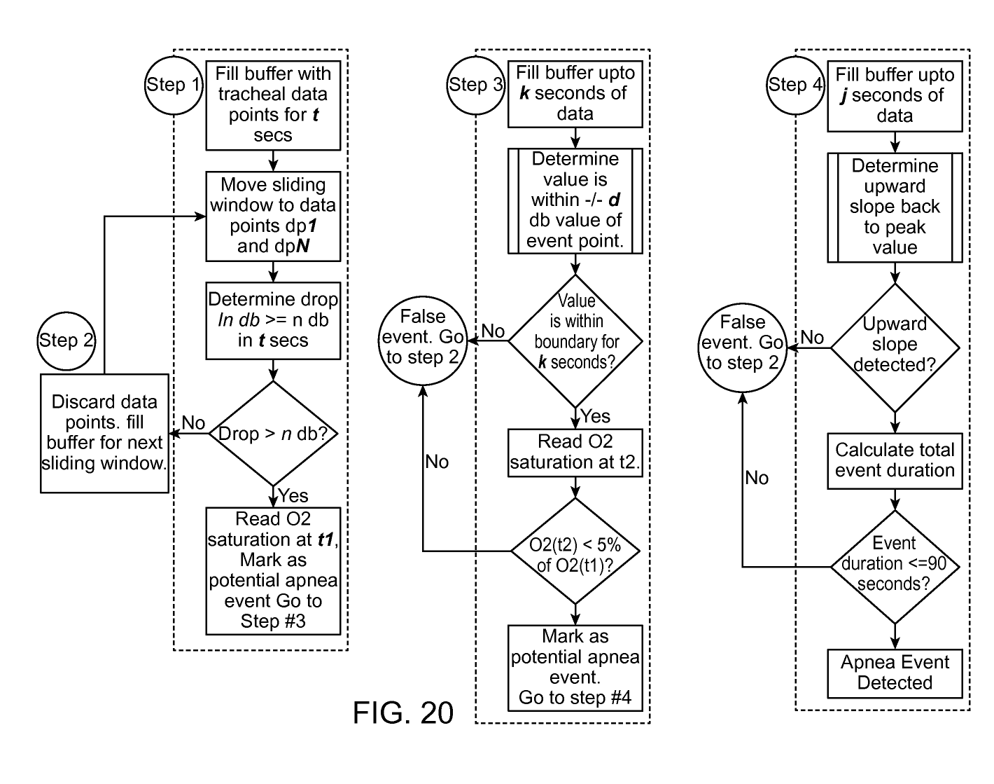

Following are the four steps in apnea detection.
1. The DSP algorithm uses the sliding time window and compares the sound intensity vs. the sliding window moving average. If a drop in the sound db detected (compared to sliding window average). Time is marked as the potential apnea event and skip step 2.
2. If there is no drop then the window average is updated with the new value.
3. Observe the sound signal if the drop lasts for certain time (apnea threshold) if yes then observe the SaO2 level. If the drop in SaO2 is more than 5% of previous value then mark as potential event and go to step 4. Otherwise go back to step 2. (FIG. 20)
4. Observe if there is upward slop in sound is detected (indicates airflow getting normal). If yes then measure the total interval time and if it is longer the typical apnea event then it is an event. Otherwise if there is not upward slop is detected then this is not an event, go back to step 2.

In the therapeutic device, the apnea detection is similar as in the diagnostic device. As soon as the onset of the apneic/snoring event is detected, the device starts an EMS response. EMS response has many possible variables to create different combinations of stimulations. These combinations can be cycled through during the testing to select the optimal combination for a patient in given conditions. Stimulation can be varied by changing following parameters;
1. Intensity
2. Pulse (Shape and Duty Cycle)
3. Frequency
4. Pattern

According to the research paper Continuous Transcutaneous Submental Electrical Stimulation in Obstructive Sleep Apnea Published in CHEST 2011; 140(4):998-1007, the maximum stimulation applied to genioglossus muscle without causing arousal or waking from sleep is 14.8 mA with SD of 6.9 Most of the patients respond to the 10.1 mA with SD 3.7 as the sufficient to contract genioglossus muscle.

Exemplary intensity variation useful in the present invention are given below:

TABLE 1
EMS Current Ranges

| | | Min | Max |
|---|---|---|---|
| Range | Steps | (mA) | (mA) |
| Low | 15 | 3 | 10 |
| Nominal | 15 | 3 | 14 |
| High 1 | 15 | 4 | 20 |
| High 2 | 15 | 5 | 25 |

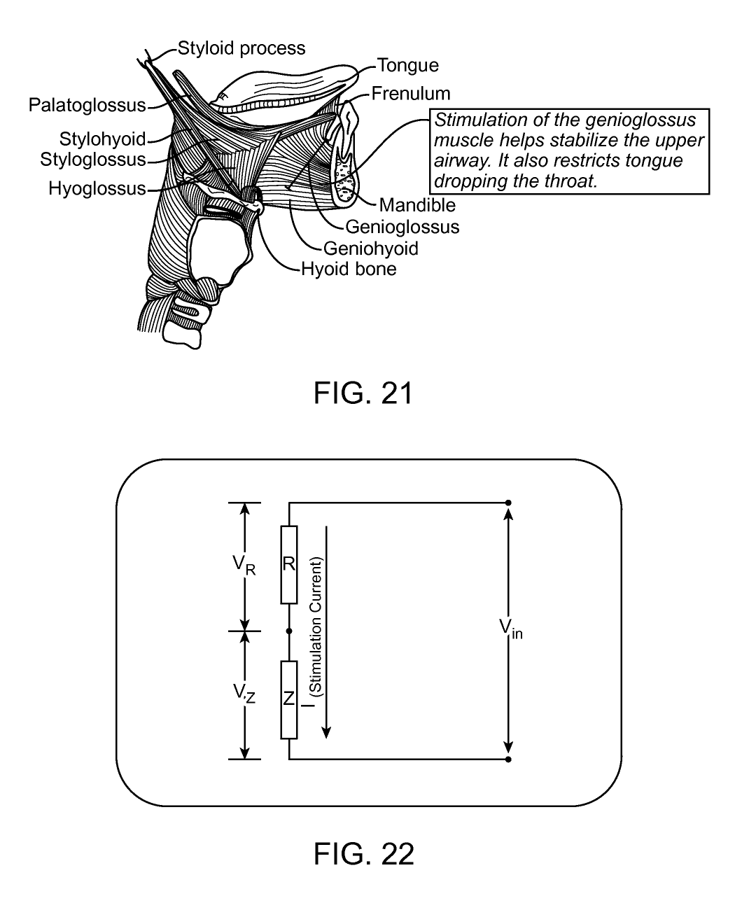

Muscle stimulation current is stabilized around the desired current needed to open the upper airway by stimulation of genioglossus muscle (muscle under the tongue). The airflow is constantly monitored via tracheal sound signal, as soon as the normal or close to normal airflow (no obstruction) is achieved the current value is recorded as the desired current for that position and condition to maintain the obstruction free breathing. (See FIG. 21).

Electric current value is determined by measuring the voltage drop across a known value series resistance “R” placed in the current stimulations path inside the device. The total voltage driving the current is adjusted accordingly to compensate any change in the total impedance of the stimulations path. (See FIG. 22).

Stimulation current intensity is controlled by the input voltage value. Since the stimulation path impedance varies depending upon the position, pad's degree of contact, body and room temperature, moisture level in the skin etc. To keep the stimulation current at the “desired level” we need to adjust the input voltage accordingly.

### Input Voltage=Vin

R=Series resistance placed in the stimulation current path
Z=Impedance of stimulation current path
(Includes pads, contact resistance, skin, fat and muscle)

### Vin=VR+VZ

$$Stimulation Current = I = \frac{Vin}{R + Z}$$

Since R is in series with Z same current flows through both of them.

If “Z” varies “Vᵢₙ” should also change accordingly to keep the stimulations current at desired levels. Once desired level of stimulation current is determined, corresponding V_{R} for that current is measured and recorded. Later we keep observing the V_{R} intermittently. Any change in V_{R} will indicate the change in Z. So to keep the current same we need Vᵢₙ should track Z.

$$Stimulation Current = I = \frac{Vin ↑}{R + Z ↑} = \frac{Vin ↓}{R + Z ↓}$$

If V_{Rnew}>V_{Rold} (It represents that the Z_{new}<Z_{old}) In this case reduce Vin to lower the current until V_{Rnew}=V_{Rold}

If V_{Rnew}<V_{Rold} (It represents that the Z_{new}>Z_{old}) In this case boost Vin to increase the current until V_{Rnew}=V_{Rold}

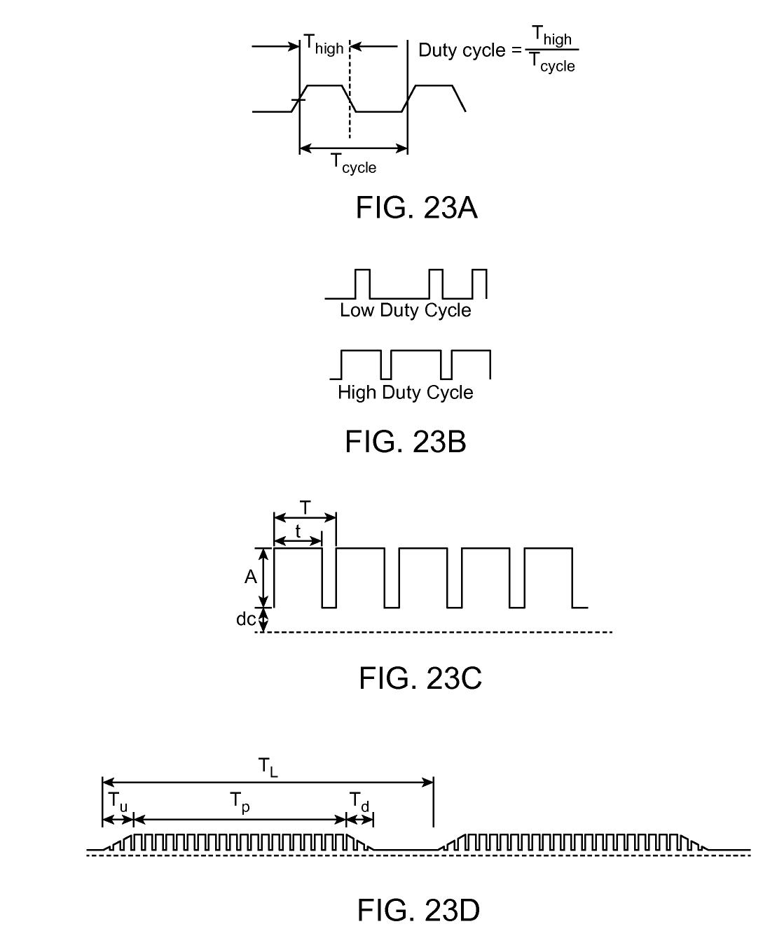

EMS can be applied continuously or in form of pulse. We can have different pulse widths or duty cycle. This feature is used on subjects in addition to the intensity as a variable to generate better response on the muscle. (Table 2; FIG. 23)

TABLE 2
Pulse Shape and Duty Cycle

| Pulse Type | Duty Cycle (%) |
|---|---|
| Square | 5 |
| | 10 |
| | 25 |
| | 50 |
| Triangle | 5 |
| | 10 |
| | 25 |
| | 50 |
| Sinusoidal | 5 |
| | 10 |
| | 25 |
| | 50 |

The nomenclature in FIGS. 23A-23D is as follows;

| | |
|---|---|
| T | Time period of a waveform |
| f | Frequency of waveform = 1/T |
| t | pulse width |
| DUTY | duty cycle = t/T × 100 |
| dc | dc offset of the waveform |
| A | amplitude of the waveform |
| Tₚ | duration of the pattern |
| Tᵤ | duration of the linear gradual increase |
| T_{d} | duration of the linear gradual decrease |
| T_{L} | duration after which the pattern repeats it self |

For the pulse stimulation options (not DC or continuous), we can vary the frequency to see the impact of the stimulation. Frequency can be used in combination of the pulse width since very high frequency may depict higher duty cycle or even continuous or DC stimulation. See Table 3.

TABLE 3

| Freq(Hz) | |
|---|---|
| Range | 40-200 |
| Low | 40 |
| Medium | 66 |
| High | 200 |

The diagnostic and therapeutic devices are each a self-learning device uses artificial intelligence to adopt and adjust to the circumstances in which it is used. Each device adjusts itself to changes in the environmental conditions during the test as well as the changes in patient/user's conditions. Built in artificial intelligence also keeps tracks of the trends and historic values. These values will be updated during each use and serve as the starting point in later in similar conditions. For example the mean stimulation current values will be recorded for each sleep position. When patient returns to the particular sleep position, stimulation current values will be adjusted to the previously recorded level. And during operation it will be fine-tuned by calibration. The different permutations of different conditions and their corresponding stimulation levels may be kept in a user history/profile.

Historic data and profile is kept on the device memory as well as in remote database (cloud). When the device is replaced or shared among users it will download the patient data if exists from the cloud to device. In case of no historic data, device will load the generic values based on the patient attributes, BMI, neck circumference, skin condition and gender.

Condition Variables Used:
1. Environmental noises
2. Circulation Vent ON/OFF, Bed partner movement, Random noises
3. Electrodes (Pads) degree of contact to patient's skin
4. Body position (Left, Right, Supine and Prone)
5. Neck rotation relative to the body
6. Snoring sound (Self and bed partner)
7. Sleep stages
8. Changes in skin condition
9. Moisture level, PH value, Sub dermal fat

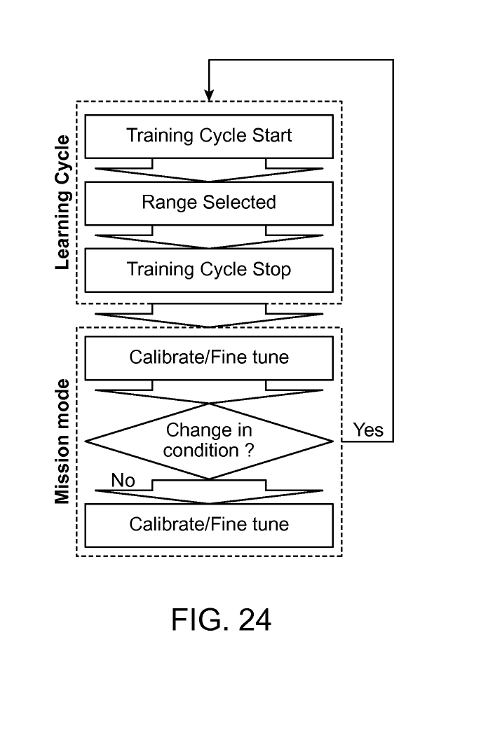

Changes in the variables are monitored during the normal operation as well as in intermittent training cycles. Macro calibration is done using dedicated training cycles and during mission modes fine-tuning or micro calibration is done. In the dedicated training cycles we chose among the major ranges. During the normal operation or mission mode fine-tuning is done within the ranges. Fine-tuning is done in small steps until a lock value is achieved. (See FIG. 24).

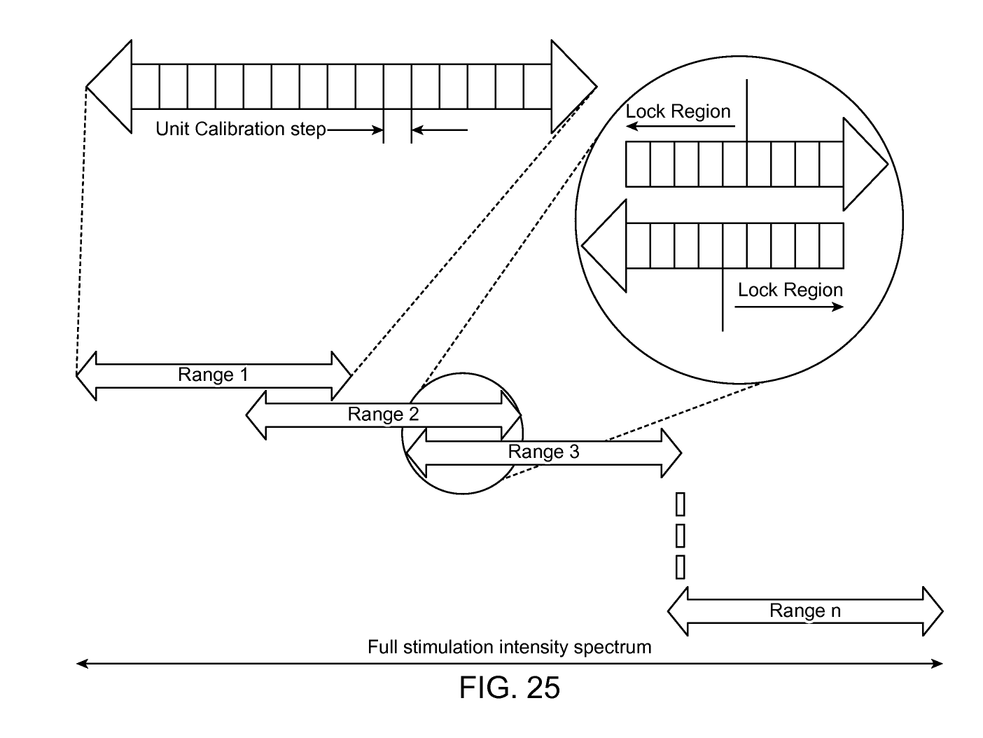

The entire spectrum is divided in several macro ranges. These ranges have an overlap between adjacent ranges. Selection among ranges is done during the learning cycle. During the training cycle device tests the impedance of entire stimulation path (leads, electrodes, electrode-skin contact, sub-dermal fat and muscle). Based on the detected impedance value the stimulation current range is selected. If the value falls in the overlap region, then we select the range that offers higher degree of calibration points. As soon as the range is selected training cycle is stopped and the normal operation starts. During the normal operation calibration within the range are done. Training cycle gives the initial lock value. (See FIG. 25)

The main purpose of the training cycles is to allow macro adjustments or select between the wide ranges. There are two types of training cycles. These training cycles happen when preset conditions are met. During this process the devices suspends normal operation (Data Collection for diagnostic and EMS response for apneic/snoring event) for very short duration of time. The device microprocessor observes and processes sensors data establishes and updates new reference values. It adjusts the initial value of the response intensity, duration and pattern. These training cycles are invoked at:
1. First use of the device
2. Anytime device is attached to user
3. At the start of sleep
4. At the resumption of sleep from full awake condition during use

These training cycles happen as device detects changes in the condition or determines the major calibration is needed. Like pre-defined training cycles these also suspends the normal operation of the ARB and adjust reference values based on the learning during training cycles. The frequency of these cycles is not defined however we can limit their re-occurrence based on adjustments in the criteria.
1. Change in body position
2. Change in sleep stage
3. Major change in conditions (e.g., Sustained environmental noise, skin moisture level change due to perspiration)
4. Device dislocation with respect to body

This calibration happens during the normal operation. The device detects the changes in the sensor data and makes minor adjustments to reference values as well as the EMS response. The magnitude of the change (delta between pre and post change values) is relatively smaller. Changes happen gradually and adjustments are made in steps.

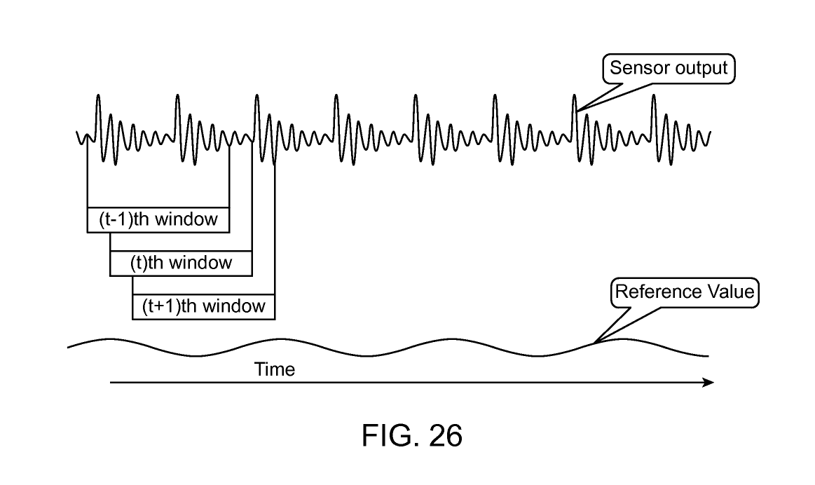

For the sensor data reference values are adjusted based the average or mean value of the given duration. The sensor inputs are buffered for the duration. Newer values replace the oldest values (Last in first out) in the sliding window. Window size is selected long enough, so we can filter out any anomalies in the data. (See FIG. 26).

EMS response intensity i.e., the stimulation current is adjusted based the current path impedance. If there is any change in the current path impedance current amplitude or intensity will has to adjust accordingly. Current path impedance can vary due to variety of different factors, skin moisture level, degree of contact between skin and the gel pads, sub dermal fat between the skin the muscle due to neck movement.

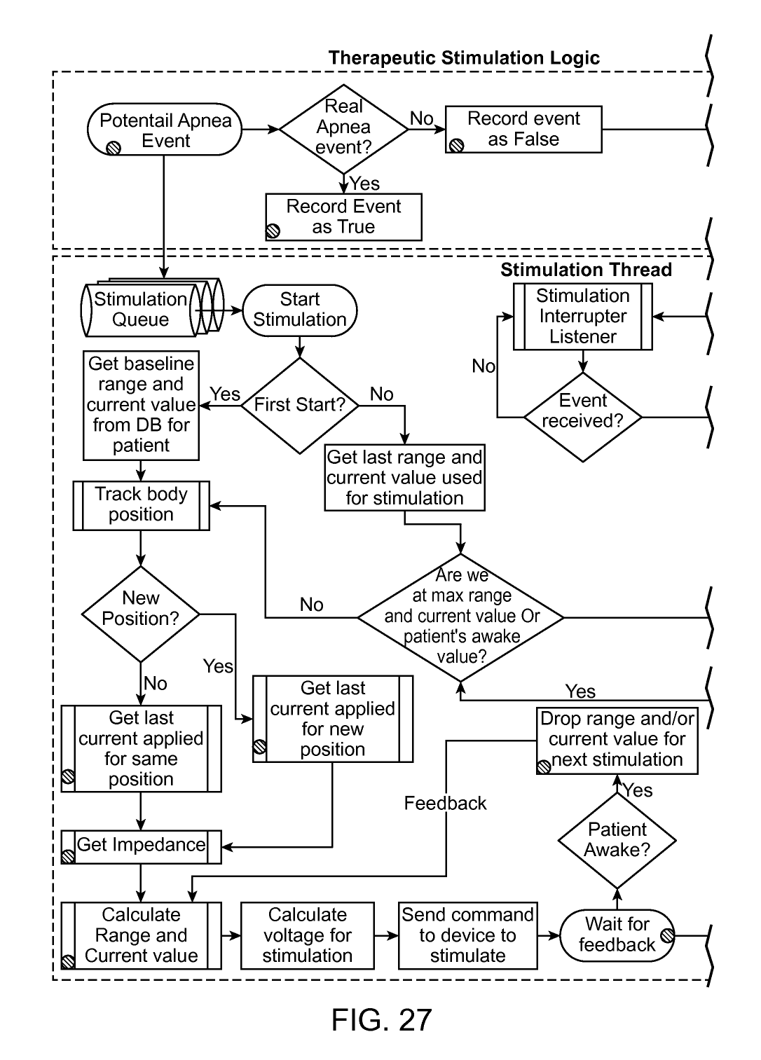

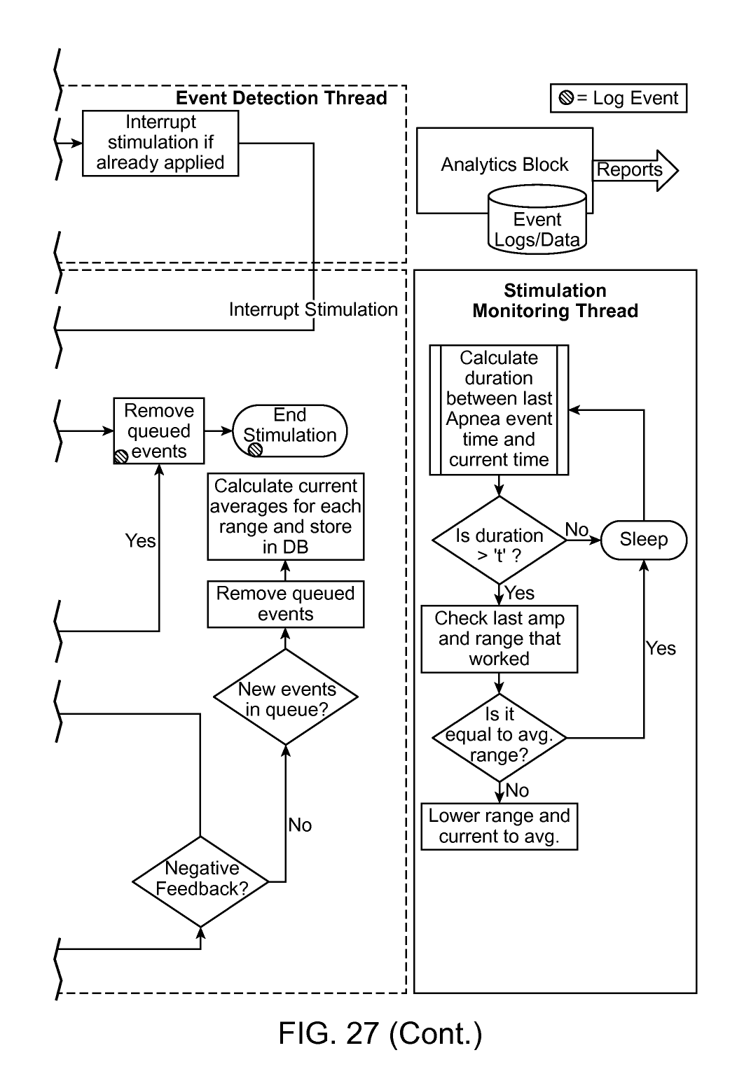

The device measures the current path impedance on regular intervals and maintains the historic average in the buffer. At the beginning of apneic/snoring event, impedance is again tested if a difference greater than the threshold is detected, and multiple measurements are enforced. If the difference from the recorded average persists than we update the average value in the buffer and use new value for the stimulation current adjustment. However, if repeated measurements are not consistent then the deviating values are dropped as false values. Stimulation current is adjusted according to the average value of the impedance based on previous and new recoded impedance. (See FIG. 27).

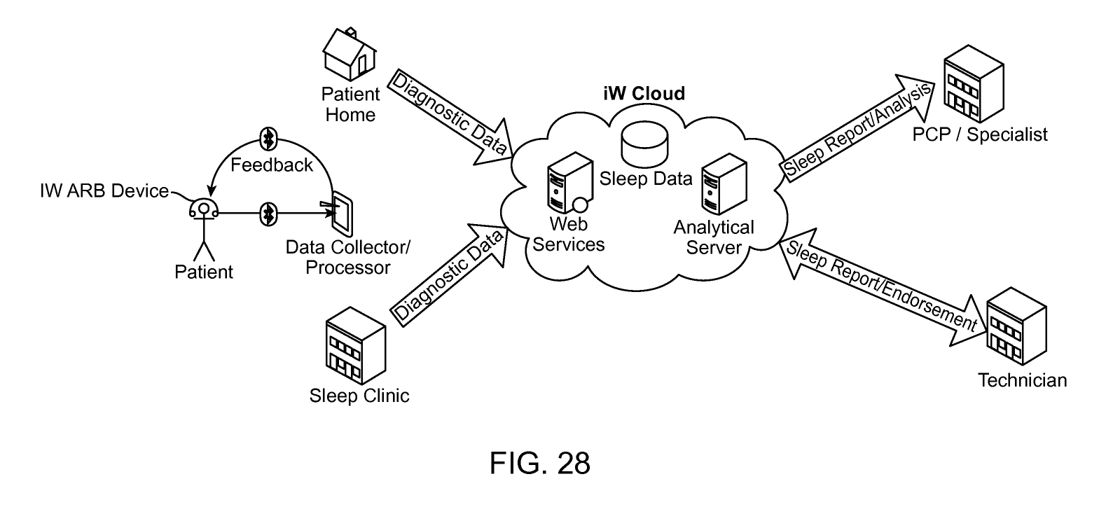

Sleep data is uploaded on the cloud. Data is encrypted to meet all of the data security requirement of HIPAA. Data is kept on the server for physician's access as well as it is relayed to the technician. Data is kept in a dedicated folder for each patient. Data is updated as the device is used. (See FIG. 28).

What is claimed is:

1. A device for collecting sleep data from a patient, said device comprising:
a component wearable by the patient;
a microprocessor, memory, and a power source on the component;
a plurality of at least two sensors connectable to the microprocessor, said sensors selected from the group consisting of:
(a) a microphone for detecting tracheal sounds;
(b) a microphone for detecting snoring sounds;
(c) a microphone for detecting ambient sounds;
(d) a pulse oximeter;
(e) a body position sensor;
(f) a body motion sensor;
(g) a breathing effort sensor;
(h) ECG electrodes;
(i) sleep stage sensors; and
(j) a muscle tone sensor;
wherein the microprocessor stores in the memory and/or analyzes at least a portion of data produced by the sensors.

2. A device as in claim 1, said device comprising at least three sensors connectable to the microprocessor.

3. A device as in claim 1, said device comprising at least four sensors connectable to the microprocessor.

4. A device as in claim 1, said device comprising at least five sensors connectable to the microprocessor.

5. A device as in claim 1, said device comprising at least six sensors connectable to the microprocessor.

6. A device as in claim 1, wherein the component comprises a neck band.

7. A device as in claim 1, wherein at least some of the sensors are disposed on the component.

8. A device as in claim 7, wherein at least some of the sensors are connected to the component by a connector element.

9. A device as in claim 8, wherein the connector element is a flexible cable.

10. A device as in claim 8, wherein the connector element is a wireless connector element.

11. A system for collecting sleep data from a patient, said system comprising:
a collection device as in claim 1; and
a remote storage and/or analytical device which receives data transmitted from the collection device.

12. A method for collecting sleep data from a patient, said method comprising:
placing a component on the patient, wherein said component carries a microprocessor, memory, and a power source; and
collecting data relating to at least two symptoms selected from the group consisting of:
(a) tracheal sounds;
(b) snoring sounds;
(c) ambient sounds;
(d) blood oxygen saturation;
(e) body position;
(f) breathing effort;
(g) ECG;
(h) sleep stage; and
(j) muscle tone;
wherein the data are collected in accordance with rules implemented by the microprocessor and stored in the memory.

13. A method as in claim 12, wherein data are collected relating to at least three symptoms.

14. A method as in claim 12, wherein data are collected relating to at least four systems.

15. A method as in claim 12, wherein data are collected relating to at least five symptoms.

16. A method as in claim 12, wherein data are collected relating to at least six symptoms.

17. A method as in claim 12, wherein the component is worn on the neck.

18. A method as in claim 12, wherein data is collected with sensors which are connected to deliver the data to the microprocessor.

19. A method as in claim 18, wherein at least some of the sensors are disposed on the component.

20. A method as in claim 18, wherein at least some of the sensors are disposed remotely from the component.

21. A method as in claim 12, further comprising transmitting the collected data to a remote storage and/or analytical device.

22. A method as in claim 21, wherein the remote storage and/or analytical device is worn or carried by the patient.

23. A method as in claim 12, wherein the remote storage and/or analytical device is maintained locally of the patient.

24. A method as in claim 12, further comprising re-transmitting at least a portion of the collected data to a central storage location.

25. A device for restoring air flow in a sleeping patient, said method comprising:
a component wearable by the patient;
a microprocessor, memory, circuitry, and a power source on the component;
at least one sensor connectable to the microprocessor to sense a patient symptom characteristic of disrupted air flow; and
at least one output element connectable to the microprocessor, said output element configured to deliver energy to the patient to restore air flow while the patient remains sleeping;
wherein the microprocessor is configured to deliver an output to the patient through the output element; and
wherein the microprocessor is configured to correlate the ability of a particular output to restore air-flow with the patient symptoms and adjust the output delivered through the output element at least partially based on such a correlation.

26. A device as in claim 25, wherein the component comprises a neck band.

27. A device as in claim 25, wherein the at least one sensor is selected from the group consisting of:
(a) a microphone for detecting tracheal sounds;
(b) a microphone for detecting snoring sounds;
(c) a microphone for detecting ambient sounds;
(d) a pulse oximeter;
(e) a body position sensor;
(f) a body motion sensor;
(g) a breathing effort sensor;
(h) ECG electrodes;
(i) sleep stage sensors; and
(j) a muscle tone sensor.

28. A device as in claim 25, wherein the output element comprises one or more electrical delivery elements.

29. A device as in claim 25, wherein the microprocessor is configured to control at least one property of an electrical output, said property being selected from the group consisting of current, voltage, power, frequency, pulse repetition pattern, pulse width, duty cycle and waveform.

30. A device as in claim 29, wherein the microprocessor and the memory are configured to store data representing the correlations between delivered outputs and ability of a delivered output to restore air flow.

31. A device as in claim 30, further comprising a transmitter configured to deliver the data to an outside receiver which can store and/or retransmit the data.

32. A method for restoring air flow in a sleeping patient, said method comprising:
monitoring at least one symptom of air flow disruption while the patient is sleeping;
applying an initial stimulating energy to a muscle of the patient's upper airway when a symptom of air flow disruption is detected;
determining whether the symptom of air flow disruption has been alleviated in response to the initial stimulating energy;
if the symptom has not been alleviated, apply additional stimulating energy to the muscle, wherein the additional stimulating energy has been adjusted to enhance effectiveness in alleviating the symptom.

33. A method as in claim 32, further comprising recording data which correlates the ability or inability of stimulating energy having particular characteristics in relieving particular symptoms to establish a baseline for treating individual patients.

34. A method as in claim 33, wherein the initial stimulating energy is selected based on a previously established baseline for the patient.

35. A method as in claim 33, further comprising locally storing the baseline data on a device used to effect the treatment.

36. A method as in claim 33, further comprising remotely storing the baseline data.

37. A method as in claim 32, wherein the at least one symptom is selected from the group consisting of:
(a) tracheal sounds;
(b) snoring sounds;
(c) ambient sounds;
(d) blood oxygen saturation;
(e) body position;
(f) breathing effort;
(g) ECG;
(h) sleep stage; and
(i) muscle tone.

38. A method as in claim 32, wherein the initial and additional stimulating energy are electrical.

39. A method as in claim 38, wherein the additional stimulating energy is adjusted in at least one of current, voltage, power, frequency, pulse width, pulse repetition, and wave form.
